(19) United States
(12) Patent Application Publication
(10) Pub. No.: US 2017/0176440 A1
(43) Pub. Date: Jun. 22, 2017

(54) METHODS AND COMPOSITIONS FOR EVALUATING BREAST CANCER PATIENTS

(71) Applicants: Rong LI, San Antonio, TX (US); Bin YUAN, San Antonio, TX (US); Qinong YE, Beijing (CN); Long CHENG, Beijing (CN)

(72) Inventors: Rong LI, San Antonio, TX (US); Bin YUAN, San Antonio, TX (US); Qinong YE, Beijing (CN); Long CHENG, Beijing (CN)

(73) Assignee: THE BOARD OF REGENTS OF THE UNIVERSITY OF TEXAS SYSTEM, Austin, TX (US)

(21) Appl. No.: 15/129,597

(22) PCT Filed: Mar. 26, 2015
(86) PCT No.: PCT/US15/22788

§ 371 Date: Sep. 27, 2016

Related U.S. Application Data
(60) Provisional application No. 61/970,942, filed on Mar. 27, 2014.

(51) Int. Cl. G01N 33/574; C07K 16/28

(52) U.S. Cl. CPC G01N 33/57415; C07K 16/2869; G01N 2800/52; C07K 2317/30

(57) ABSTRACT

Embodiments are directed to methods of evaluating the prognosis of a breast cancer patient by assessing the level of phosphorylation tyrosine 36 of estrogen receptor beta (ERB). Certain embodiments are directed to methods for evaluating the prognosis of a cancer patient comprising contacting a breast cancer sample from a patient with stage II or stage III breast cancer with an antibody that binds phosphorylated tyrosine 36 of Ei¾β; quantifying phosphorylation of tyrosine 36 of EI¾β; and classifying the patient as having a good prognosis if phosphorylated tyrosine 36 levels are elevated or classifying the patient as having a poor prognosis if tyrosine levels are decreased relative to a non-cancer control. Certain embodiments are directed to an antibody or antibodies that specifically bind tyrosine 36 (Y36) of the ERp.

### REFERENCE TO SEQUENCE LISTING

This application claims priority to U.S. Provisional Patent application Ser. No. 61/970,942 filed Mar. 27, 2014, which is incorporated herein by reference in its entirety.

A sequence listing required by 37 CFR 1.821-1.825 is being submitted electronically with this application. The sequence listing is incorporated herein by reference.

### STATEMENT REGARDING FEDERALLY FUNDED RESEARCH

This invention was made with government support under CA161349 awarded by the National Cancer Institute. The government has certain rights in the invention.

### BACKGROUND

The two estrogen receptors, ERα and ERβ, mediate diverse effects of estrogens in multiple tissues (Thomas and Gustafsson, Nature Rev Cancer, 2011, 11(8):597-608). Despite considerable sequence homology, ERα and ERβ carry out nonredundant physiological functions. While ERα is critical for mediating estrogen-dependent proliferation during normal mammary gland development, ERβ is known to inhibit cell proliferation and promote differentiation in a number of tissues (Thomas and Gustafsson, Nature Rev Cancer, 2011, 11(8):597-608; Deroo and Buensuceso, Mol Endocrinol, 2010, 24(9):1703-14). In cancer development and progression, ERα has a well-established role in supporting estrogen-dependent breast tumor growth, whereas ERβ significantly attenuates cell proliferation and invasion in a number of cancer cell types including breast (Hartman et al., Cancer Res., 2006, 66(23):11207-13; Mak et al., Neoplasia., 2006, 8(11):896-904; Hodges-Gallagher et al., Breast Cancer Res Treat., 2008, 109(2):241-50; Thomas et al., Breast Cancer Res., 2012, 14(6):R148) and prostate cancers (Nanni et al., J Clin Invest., 2009, 119(5):1093-108; Mak et al., Cancer Cell, 2010, 17(4):319-32; Nakajima et al., Sci Signal., 2011, 4(168):ra22). Lower expression of ERβ is found in breast cancer and it correlates with worse disease outcome (Gallo et al., Curr Pharm Des. 2012, 18(19):2734-57). The fact that ERβ is still present in a large percentage of breast tumors raises the possibility of mobilizing the antitumor activity of ERβ as a potential therapy (Marotti et al., Modern Pathology, 2010, 23(2):197-204). However, this opportunity has not been extensively exploited, partly due to the paucity in the knowledge of how such ERβ activity can be harnessed in tumor cells.

Mammalian eye absent (EYA) proteins are involved in cell-fate determination in a broad spectrum of cells and tissues (Hanson, Semin Cell Dev Biol., 2001, 12(6):475-84). EYA proteins are transcription coregulators with a well-documented tyrosine phosphatase activity (Rayapureddi et al., Nature, 2003, 426(6964):295-8; Li et al., Nature, 2003, 426(6964):247-54; Tootle et al., Nature, 2003, 426(6964):299-302). The phosphatase activity of EYA is important for its roles in transcriptional regulation (Li et al., Nature, 2003, 426(6964):247-54; Ohto et al., Mol Cell Biol., 1999, 19(10):6815-24), cytoplasmic signaling (Xiong et al., Dev Cell, 2009, 16(2):271-9), innate immune response (Okabe et al., Nature, 2009, 460(7254):520-4), and DNA damage-induced apoptosis (Cook et al., Nature, 2009, 458(7238):591-6; Krishnan et al., J Biol Chem., 2009, 284(24):16066-70). An oncogenic activity of EYA proteins has been demonstrated in ovarian (Zhang et al., Cancer Res., 2005, 65(3):925-32) and breast cancers (Pandey et al., Oncogene, 2010, 29(25):3715-22; Farabaugh et al., Oncogene, 2012, 31(5):552-62). In particular, EYA2 was shown to promote proliferation, migration, and invasion of breast cancer cells, but its direct target(s) in tumor promotion is unclear.

The oncogenic activity of Bcr-Abl due to chromosomal translocation in chronic myelogenous leukemia (CML) has been extensively investigated (Colicelli, Sci Signal, 2010, 3(139):re6), and pharmacological inhibition of the c-Abl kinase activity represents one of the most successful rationale design based cancer therapies (Hunter, J Clin Invest., 2007, 117(8):2036-43). However, the function of native c-Abl protein in solid tumor development remains controversial (Ganguly and Plattner, Genes & Cancer, 2012, 3(5-6):414-25). In the case of breast cancer, c-Abl was reported to promote survival and motility of breast cancer cells (Srinivasan et al., Oncogene, 2008, 27(8):1095-105; Kiely et al., J Biol Chem., 2009, 284(30):20263-74). On the other hand, c-Abl was shown to mediate the tumor-suppressor activity of EphB4 (Noren et al., Nature Cell Biol., 2006, 8(8):815-25) and inhibit oncogenic transforming growth factor-β signaling (Allington et al., FASEB J., 2009, 23(12):4231-43) in breast tumorigenesis. Furthermore, recent clinical trials of c-Abl antagonists for several solid tumor types, including breast cancer, yielded mixed results (Cristofanilli et al., Ann Oncol., 2008, 19(10):1713-9; Yardley et al., Clin Breast Cancer, 2009, 9(4):237-42). Therefore, the exact role of c-Abl in cancer development and progression is likely to be context-dependent.

There remains a need for additional prognostic methods and compositions for evaluating breast cancer patients.

### SUMMARY

Estrogen receptors ERα and ERβ share considerable sequence homology yet exert opposite effects on breast cancer cell proliferation. In contrast to extensive studies of the proliferative role of ERα, little is known about how the antitumor activity of ERβ can be mobilized in breast cancer cells. A phosphotyrosine residue (pY36) unique to ERβ dictates ERβ-specific activation of transcription and inhibition of cancer cell growth in vitro and in vivo. Studies described herein reveal that the c-Abl tyrosine kinase and EYA2 phosphatase directly and diametrically control pY36 status and ERβ function. A non-phosphorylatable and transcriptionally active mutant of ERβ retains its antitumor activity but circumvents the control by its upstream regulators. Mechanistically, pY36 promotes ERβ-mediated coactivator recruitment to the ERβ target promoters. Consistent with the functional relationship between ERβ and its regulators, high pY36 correlates with high c-Abl but low EYA2 levels in breast cancer samples. Furthermore, compared to total ERβ, the pY36-specific ERβ signal is more strongly associated with both disease-free and overall survival in Stage II and III disease. This previously unrecognized signaling circuitry for regulation of ERβ-specific antitumor activity offers new prognostic tools and molecular targets for cancer therapy.

Certain embodiments are directed to methods for evaluating the prognosis of a cancer patient comprising contacting a breast cancer sample from a patient with stage II or stage III breast cancer with an antibody that binds phosphorylated tyrosine 36 of estrogen receptor beta (ERβ)(SEQ ID NO:1); quantifying phosphorylation of tyrosine 36 of ERβ; and classifying the patient as having a good prognosis if phosphorylated tyrosine 36 levels are elevated or classifying the patient as having a poor prognosis if tyrosine levels are decreased relative to a non-cancer control. In certain aspects the method further comprising measuring EYA2 or c-Abl activity or protein levels. In certain aspects cancer patient is a human. In a further aspect breast cancer sample is a resected tumor, an aspirate, or a biopsy sample. In certain aspects the method further comprises assessing the patient's clinical information. The patient's clinical information can include genotype, tumor size, tumor grade, lymph node status, and/or family history. The method for evaluating the prognosis of a breast cancer patient further comprises analyzing Her-2 expression levels in and/or estrogen receptor or progesterone receptor status of the breast cancer sample. Other cancers also express ERβ, these cancers (e.g., prostate, skin, and colon) can also be assed by the described methods.

In certain aspects a sample is assigned a score according to the intensity of the nucleic and/or cytoplasmic staining (no staining=0; weak staining=1, moderate staining=2, strong staining=3) and the extent of stained cells (0%=0, 1-24%=1, 25-49%=2, 50-74%=3, 75-100%=4). In certain aspects an immunoreactive score is determined by multiplying the intensity score with the extent of score of stained cells, ranging from 0 (the minimum score) to 12 (the maximum score). A score of 0 can be defined as total ERβ-negative, pY36-negative, and EYA2-negative. A score of greater than 1 is defined as ERβ positive, pY36-positive, and EYA2-positive. Furthermore, a score between 0-6 is c-Abl-negative and greater than 6 as c-Abl-positive. A score of 1 or greater indicates a good prognosis and a score of 0 indicates a poor prognosis. In certain aspects a patient with a good prognosis is also treated with one or more of an ERβ agonist (e.g., s-equol), c-abl agonist, or an EYA2 inhibitor.

Certain embodiments are directed to an antibody or antibodies that specifically bind tyrosine 36 (Y36) of the ERβ. In certain aspects the antibody or antibodies specifically bind a phosphopeptide having the amino acid sequence SIYIPSS(pY)VDSHHE (SEQ ID NO:2). In a further aspect the antibody or antibodies distinguish between ERβ proteins being phosphorylated or unphosphorylated at Y36. Certain embodiments are directed to a kit comprising an antibody or antibodies that specifically an epitope comprising a phosphor-tyrosine 36 of ERβ.

The term “breast cancer” refers to medical condition classified by biopsy of the breast as malignant pathology. One of skill in the art will appreciate that breast cancer refers to any malignancy of the breast tissue, including, for example, carcinomas and sarcomas. In particular embodiments, the breast cancer is ductal carcinoma in situ (DCIS), lobular carcinoma in situ (LCIS), or mucinous carcinoma. Breast cancer also refers to infiltrating ductal (IDC) or infiltrating lobular carcinoma (ILC). In most embodiments of the invention, the subject of interest is a human patient suspected of or actually diagnosed with breast cancer.

The American Joint Committee on Cancer (AJCC) has developed a standardized system for breast cancer staging using a “TNM” classification scheme. Patients are assessed for primary tumor size (T), regional lymph node status (N), and the presence/absence of distant metastasis (M) and then classified into stages 0-IV based on this combination of factors. In this system, primary tumor size is categorized on a scale of 0-4 (T0: no evidence of primary tumor; T1: ≦2 cm; T2:>2 cm-≦5 cm; T3:>5 cm; T4: tumor of any size with direct spread to chest wall or skin). Lymph node status is classified as N0-N3 (N0: regional lymph nodes are free of metastasis; N1: metastasis to movable, same-side axillary lymph node(s); N2: metastasis to same-side lymph node(s) fixed to one another or to other structures; N3: metastasis to same-side lymph nodes beneath the breastbone). Metastasis is categorized by the absence (M0) or presence of distant metastases (M1). Methods of identifying breast cancer patients and staging the disease are well known and may include manual examination, biopsy, review of patient's and/or family history, and imaging techniques, such as mammography, magnetic resonance imaging (MRI), and positron emission tomography (PET).

Once the T, N, and M categories have been determined, this information is combined in a process called stage grouping. Cancers with similar stages tend to have a similar outlook and are often treated in a similar way. Stage is expressed in Roman numerals from stage I (the least advanced stage) to stage IV (the most advanced stage). Non-invasive cancer is listed as stage 0.

Stage 0: Tis, N0, M0: This is ductal carcinoma in situ (DCIS), a pre-cancer of the breast. In all cases the cancer has not spread to lymph nodes or distant sites.

Stage IA: T1, N0, M0: The tumor is 2 cm (about ¾ of an inch) or less across (T1) and has not spread to lymph nodes (N0) or distant sites (M0).

Stage IB: T0 or T1, N1mi, M0: The tumor is 2 cm or less across (or is not found) (T0 or T1) with micrometastases in 1 to 3 axillary lymph nodes (the cancer in the lymph nodes is greater than 0.2 mm across and/or more than 200 cells but is not larger than 2 mm)(N1mi). The cancer has not spread to distant sites (M0).

Stage II cancer includes stage IIA and IIB.

Stage IIA: One of the following applies: (a) T0 or T1, N1 (but not N1mi), M0: The tumor is 2 cm or less across (or is not found) (T1 or T0) and either: It has spread to 1 to 3 axillary lymph nodes, with the cancer in the lymph nodes larger than 2 mm across (N1a), or Tiny amounts of cancer are found in internal mammary lymph nodes on sentinel lymph node biopsy (N1b), OR It has spread to 1 to 3 lymph nodes under the arm and to internal mammary lymph nodes (found on sentinel lymph node biopsy) (N1c). (b) T2, N0, M0: The tumor is larger than 2 cm but less than 5 cm across (T2) but hasn't spread to the lymph nodes (N0). The cancer hasn't spread to distant sites (M0).

In stage IIB: One of the following applies: (a) T2, N1, M0: The tumor is larger than 2 cm but less than 5 cm across (T2). It has spread to 1 to 3 axillary lymph nodes and/or tiny amounts of cancer are found in internal mammary lymph nodes on sentinel lymph node biopsy (N1). The cancer hasn't spread to distant sites (M0). (b) T3, N0, M0: The tumor is larger than 5 cm across but does not grow into the chest wall or skin and has not spread to lymph nodes (T3, NO). The cancer hasn't spread to distant sites (M0).

Stage III includes stage IIIA, stage IIIB, and stage IIIC.

Stage IIIA: One of the following applies: T0 to T2, N2, M0: The tumor is not more than 5 cm across (or cannot be found) (T0 to T2). It has spread to 4 to 9 axillary lymph nodes, or it has enlarged the internal mammary lymph nodes (N2). The cancer hasn't spread to distant sites (M0); or T3, N1 or N2, M0: The tumor is larger than 5 cm across but does not grow into the chest wall or skin (T3). It has spread to 1 to 9 axillary nodes, or to internal mammary nodes (N1 or N2). The cancer hasn't spread to distant sites (M0).

Stage IIIB: T4, N0 to N2, M0: The tumor has grown into the chest wall or skin (T4), and one of the following applies: It has not spread to the lymph nodes (N0). It has spread to 1 to 3 axillary lymph nodes and/or tiny amounts of cancer are found in internal mammary lymph nodes on sentinel lymph node biopsy (N1). It has spread to 4 to 9 axillary lymph nodes, or it has enlarged the internal mammary lymph nodes (N2). The cancer hasn't spread to distant sites (M0). Inflammatory breast cancer is classified as T4d and is at least stage IIIB. If it has spread to many nearby lymph nodes (N3) it could be stage IIIC, and if it has spread to distant lymph nodes or organs (M1) it would be stage IV.

Stage IIIC: any T, N3, M0: The tumor is any size (or can't be found), and one of the following applies: Cancer has spread to 10 or more axillary lymph nodes (N3). Cancer has spread to the lymph nodes under the clavicle (collar bone) (N3). Cancer has spread to the lymph nodes above the clavicle (N3). Cancer involves axillary lymph nodes and has enlarged the internal mammary lymph nodes (N3). Cancer has spread to 4 or more axillary lymph nodes, and tiny amounts of cancer are found in internal mammary lymph nodes on sentinel lymph node biopsy (N3). The cancer hasn't spread to distant sites (M0).

Stage IV: any T, any N, M1: The cancer can be any size (any T) and may or may not have spread to nearby lymph nodes (any N). It has spread to distant organs or to lymph nodes far from the breast (M1). The most common sites of spread are the bone, liver, brain, or lung,

The term “prognosis” is recognized in the art and encompasses predictions about the likely course of disease or disease progression, particularly with respect to likelihood of disease remission, disease relapse, tumor recurrence, metastasis, and death. “Good prognosis” refers to the likelihood that a patient afflicted with cancer, particularly breast cancer, will remain disease-free (i.e., cancer-free). “Poor prognosis” is intended to mean the likelihood of a relapse or recurrence of the underlying cancer or tumor, metastasis, or death. Cancer patients classified as having a “good outcome” remain free of the underlying cancer or tumor. In contrast, “bad outcome” cancer patients experience disease relapse, tumor recurrence, metastasis, or death. In particular embodiments, the time frame for assessing prognosis and outcome is, for example, less than one year, one, two, three, four, five, six, seven, eight, nine, ten, fifteen, twenty, or more years. As used herein, the relevant time for assessing prognosis or disease-free survival time begins with the surgical removal of the tumor or suppression, mitigation, or inhibition of tumor growth. Thus, for example, in particular embodiments, a “good prognosis” refers to the likelihood that a breast cancer patient will remain free of the underlying cancer or tumor for a period of at least five, such as for a period of at least ten years. In further aspects of the invention, a “poor prognosis” refers to the likelihood that a breast cancer patient will experience disease relapse, tumor recurrence, metastasis, or death within less than ten years, such as less than five years. Time frames for assessing prognosis and outcome provided herein are illustrative and are not intended to be limiting.

In particular embodiments, ERβ-pY36 is statistically significant for assessment of the likelihood of breast cancer recurrence or death due to the underlying breast cancer. Methods for assessing statistical significance are well known in the art and include, for example, using a log-rank test, Cox analysis and Kaplan-Meier curves. In some aspects of the invention, a p-value of less than 0.05 constitutes statistical significance.

Certain embodiments are directed to methods for assessing or monitoring the effectiveness of a cancer treatment comprising contacting a breast cancer sample from a patient with stage II or stage III breast cancer that has been administered an anti-cancer therapy with an antibody that binds phosphorylated tyrosine 36 of estrogen receptor β; quantifying phosphorylation of tyrosine 36 of ERβ; and classifying the treatment as effective if phosphorylated tyrosine 36 levels are elevated relative to levels prior to treatment. In certain aspects a sample taken prior to treatment is assessed using the methods described herein. In other aspects, samples taken at various time points during treatment are assessed the methods described herein.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. EYA2 modulates the transcriptional activity of ERβ, but not ERα. (A) Co-IP of endogenous EYA2 and ERβ in a sub-strain of MCF7 breast cancer cell line that expresses both ER proteins. 17β-estrodial (E2; 10 nM) was used. (B) GST-EYA2 pull-down with in vitro translated ERβ in the presence of vehicle or various ligands. 5% input protein was loaded. An ERα-specific agonist propyl-pyrazole triol (PPT; 1 nM) or an ERβ-specific agonist diarylpropionitrile (DPN; 10 nM) was used. (C) Real-time RT-PCR assesses the effect of EYA2 overexpression on ERβ-mediated transcription of its target genes MDA7 and pS2 in MCF7 cells. The value for column 1 is set as 1. (D) Effects of EYA2 knockdown on ERβ-mediated transcription of MDA7 and pS2 in MCF7 cells. * p<0.05; ** p<0.01. Gel images in this and following figures are representatives of at least three independent experiments. Graphs throughout the figures are average of at least three experiments. Error bars represent standard error of the mean (s.e.m.).

FIG. 2. EYA2 modulates the transcriptional activity of ERβ, but not ERα. (A) ERβ immunoblot using lysates from parental and siRNA knockdown cells, as well as primary breast cancer (BC) tissue. (B) EYA2 represses ligand-stimulated transcription of ERβ target genes in MDA-MB-231 cells. Shown on the right is the EYA2 immunoblot using lysates from control and ectopic EYA2-expressing MDA-MB-231 cells, as well as primary breast cancer tissue. ERβ-specific agonist diarylpropionitrile (DPN; 10 nM) was used. (C) Real-time RT-PCR assesses the effect of EYA2 overexpression on ERβ-mediated transcription of its target genes MDA7 and MSMB in HEK293T cells. The value for ER-transfected cells without E2 and EYA2 is set as 1. (D) Effects of EYA2 knockdown on ERβ-mediated transcription of MDA7 and MSMB in HEK293T cells. (E) EYA2 overexpression does not inhibit ERα-mediated activation of pS2 and GREB1 in HEK293T cells. * p<0.05; ** p<0.01. Gel images in this and following figures are representatives of at least three independent experiments. Graphs throughout the figures are average of at least three experiments. Error bars represent standard error of the mean (s.e.m.).

FIG. 3. EYA2 represses ERβ-mediated transcription of multiple ERβ target genes. (A) Immunoblots of doxycycline (dox)-inducible Flag-ERα or ERβ in Hs578T cells. (B) Verification of the ligand-stimulated transcription of ERβ target genes in the ERα or ERβ-inducible Hs578T cells. (C) Immunoblots of dox-inducible ERβ in Hs578T cells with and without ectopic EYA2. (D) ChIP verifies ligand-stimulated chromatin binding of ERβ to its target promoters. (E) EYA2 expression in ERβ-inducible Hs578T cells specifically attenuates ERβ action at the five ERβ-specific genes. The mRNA level of each gene in the presence of ERβ and absence of E2 and EYA2 is set as 1. * P<0.05; ** P<0.01. GAPDH is used as a loading control. Error bars represent standard error of the mean (s.e.m.).

FIG. 4. EYA2 inhibits ERβ transcriptional activity by directly dephosphorylating pY36. (A) Sequences of mammalian ERα and ERβ orthologs surrounding the Y36 residue of human ERβ. ClustalW was used for the sequence alignment. (B) The anti-pY36 antibody recognizes WT-ERβ, but not the Y36F mutant or ERα in IP-Western of Flag-ER proteins from HEK293T cells. (C) IP-Western of Flag-ERβ in HEK293T cells indicates that EYA2 reduces the pY36 signal. (D) EYA2 knockdown in HEK293T cells increases the pY36 signal of Flag-ERβ. (E) Ligand-stimulated pY36 signal of endogenous ERβ in MDA-MB-231 cells is reduced by EYA2. EYA2-transfected cells were treated with vehicle, E2, or DPN for 2 hr. The lysates were used in an ERβ-specific IP, followed by immunoblotting with the anti-pY36 or anti-total ERβ antibody. (F) Recombinant WT-EYA2, but not phosphatase-deficient mutant proteins, efficiently dephosphorylates immunoprecipitated Flag-ERβ in vitro. (G) Real-time RT-PCR compares WT-ERβ and the mutants in activation of ERβ target genes (MDA7 and MSMB) in MDA-MB-231 cells. Error bars represent standard error of the mean (s.e.m.). * p<0.05. ** p<0.01.

FIG. 5. EYA2 specifically dephosphorylates ERβ, not ERα. (A) Sequences of the EYA2 regions surrounding the mutated amino acid residues. (B) In vitro phosphatase assay using recombinant GST fused with WT and mutant EYA2 proteins. A pY142-H2AX peptide, previously demonstrated to be a target of EYA2 tyrosine phosphatase activity, was used as the substrate. Also shown on the top is the Coomassie Brilliant Blue-stained SDS-PAGE gel of the purified GST-EYA2 fusion proteins. (C) Real-time RT-PCR compares the effects of WT and mutant EYA2 proteins on ERβ-mediated transcriptional activation of MDA7 in HEK293T cells. The immunoblot shows the expression of the Flag-tagged EYA2 proteins with an anti-FLAG antibody. (D) EYA2 reduces the total tyrosine phosphorylation (pY) level of Flag-ERβ, but not Flag-ERα. HEK293T cells were transfected with Flag-ER plasmids, with or without EYA2. Immunoprecipitated Flag-ER proteins were probed with a pan-pY antibody.

FIG. 6. EYA2 directly dephosphorylates pY36-ERβ. (A) ERE-luciferase assay in HEK293T cells with co-transfected EYA2 and various ERβ deletion constructs. Error bars represent standard error of the mean (s.e.m.). (B) IP-Western of Flag-ERβ with alanine substitution at various tyrosine residues in AF1. Proteins were expressed in HEK293T cells, and the overall pY signal of ERβ was assessed with a pan-pY antibody. (C) Change of Y36 to phenylalanine (Y36F) results in a significant decrease in overall ERβ tyrosine phosphorylation. (D) A pY36-containing ERβ peptide is effectively dephosphorylated in vitro by recombinant WT-EYA2. A pY containing H2AX peptide was used as a positive control. (E) IP-Western using an ERα/β-positive MCF7 cell line demonstrates ligand-stimulated pY36 of endogenous ERβ. (F) Immunoblot comparing the levels of endogenous ERβ in two normal breast tissue samples with endogenous ERβ in MDA-MB-231 cells, and overexpressed Myc-ERβ in the same cell line.

FIG. 7. c-Abl directly phosphorylates Y36 and promotes ERβ-mediated transcriptional activation. (A) WT c-Abl, not a kinase-dead mutant, increases total pY and pY36-specific signals of Flag-ERβ in HEK293T cells. Immunoblotting with an anti-c-Abl antibody indicates the physical association of c-Abl and ERβ. (B) pY36 of Flag-ERβ is reduced by imatinib but enhanced by DPH, a c-Abl activator, in HEK293T cells. (C) c-Abl knockdown reduces pY36 of endogenous ERβ in MDA-MB-231 cells. (D) WT-ERβ is directly phosphorylated in vitro by purified WT, but not mutant, c-Abl. (E) c-Abl knockdown reduces transcription of ERβ target genes MDA7 and MSMB in MDA-MB-231 cells, which is rescued by an siRNA-resistant c-Abl expression vector. (F) c-Abl knockdown in MDA-MB-231 cells abolishes transcriptional activation of MDA7 and MSMB by WT-ERβ, but not the non-phosphorylatable Y36E mutant. Also included is the Y36F mutant. Error bars represent standard error of the mean (s.e.m.). * p<0.05. Error bars represent standard error of the mean (s.e.m.).

FIG. 8. c-Abl binding to ERβ, purification of WT and mutant c-Abl proteins. (A) IP of c-Abl and immunoblotting of ERβ in MDAMB-231 cells. (B) Reciprocal co-IP of the two proteins in the same cell line. (C) A doxcycline-induced expression system for Flag-tagged WT and kinase-dead mutant c-Abl. Crude lysates from uninduced and induced cells were analyzed by immunoblotting. (D) A silver-stained SDS-PAGE gel illustrates the affinity-purified Flag-tagged WT and kinase-dead mutant c-Abl (as indicated by asterisks).

FIG. 9. pY36 promotes interaction between ERβ and its coactivator. (A) Co-IP in HEK293T cells between Flag-ERβ proteins and endogenous p300. Shown on the right is normalized quantification of the immunoblots from three independent experiments. (B and C) p300 ChIP at the pY36-dependent ERβ target promoters MDA7 (B) and MSMB (C), using chromatin from Hs578T cells that expresses either Flag-tagged WT or mutant Y36F-ERβ. In all ChIP experiments, E2 (10 nM) was added to the estrogen-deprived cells. (D and E) Flag-ERβ ChIP at the MDA7 (D) and MSMB (E) promoters. (F and G) p300 ChIP at the same ERβ target promoters with or without EYA2 overexpression. Error bars represent standard error of the mean (s.e.m.). * p<0.05. ** p<0.01.

FIG. 10. pY36 is important for the antitumor activity of ERβ. (A and B) Xenograft tumor growth derived from MDA-MB-231 cells that contained empty vector (EV), WT, or mutant ERβ. The inset shows expression of the Myc-ERβ proteins and images of individual tumors upon harvest. (C) EYA2 overexpression in MDA-MB-231 cells neutralizes the antitumor activity of WT, but not Y36E mutant, ERβ. (D) Y36E mutant is more resistant to c-Abl knockdown than WT-ERβ in the xenograft tumor model. Error bars represent standard error of the mean (s.e.m.). * p<0.05.

FIG. 11. Effects of pY36, EYA2 and c-Abl on tumor cell growth. (A and B) In vitro cell growth of MDA-MB-231 cells with Flag-WT, Y36F (A) or Y36E (B) mutant ERβ. (C and D) In vitro (C) and in vivo (D) growth of MDA-MB-231 cells with and without EYA2 overexpression. (E and F) In vitro (E) and in vivo (F) growth of MDA-MB-231 cells with and without c-Abl knockdown. Error bars represent standard error of the mean (s.e.m.). * P<0.05.

FIG. 12. pY36 is important for the antitumor activity of ERβ. (A and B) Kaplan-Meier survival curves for mice that were injected with MDA-MB-231 cells that contained empty vector (EV), WT, or mutant ERβ. (C) Kaplan-Meier survival curves for mice injected with EYA2-overexpressed MDA-MB-231 cells. (D) Kaplan-Meier survival curves for mice injected with c-Abl knockdown MDA-MB-231 cells. * p<0.05.

FIG. 13. EYA2 and c-Abl regulate the antitumor activity of ERβ through pY36. (A and B) In vitro (A) and in vivo (B) growth of MDA-MB-231 cells that expressed empty vector, WT-ERβ, Y36E-ERβ, with and without EYA2 overexpression. (C and D) In vitro (C) and in vivo (D) growth of MDA-MB-231 cells that expressed empty vector, WT-ERβ, Y36E-ERβ, with and without c-Abl knockdown. Error bars represent standard error of the mean (s.e.m.). * P<0.05.

FIG. 14. Clinical correlation of pY36 in breast cancer. (A) Expression of pY36, c-Abl, and EYA2 in human breast cancer tissues. Representative immunohistochemical staining of pY36, c-Abl, and EYA2 is shown on the left. Original magnification, ×20. Scale bar: 100 μm. A summary of 104 breast cancer tissues is shown on the right, with tissues categorized by low and high expression of pY36, c-Abl, and EYA2. The P value is generated using the ×2 test. (B) Kaplan-Meier estimate of disease-free survival and overall survival in a total of 726 available specimens from the TMA (left) and the Stage II & III specimens from the TMA (right), stained with the pY36 antibody. Marks on graph lines represent censored samples. (C) Kaplan-Meier estimate of disease-free survival and overall survival in the total (left) and the Stage II and III specimens (right), stained for total ERβ.

FIG. 15. Validation of the specificity of the antibodies used in IHC. (A) Neutralization of the c-Abl IHC signal with recombinant GST-c-Abl(932-1130). Original magnification, ×20. Scale bar, 100 μm. (B) Neutralization of the EYA2 IHC signal with recombinant GST-EYA2(136-268). Scale bar, 100 μm. (C) Neutralization of the pY36 IHC signal by a pY36-containing peptide, not its unphosphorylated counterpart. Scale bar, 100 μm. (D) IP-Western of pY36 in control and ERβ-knockdown MDA-MB-231 cells. (E) Immunocytochemistry of embedded control and ERβ knockdown MDA-MB-231 cells with the anti-pY36 antibody. Scale bars, 100 and 20 μm for the low (×10) and high (×40) magnifications, respectively.

FIG. 16. pY36 correlates with patient survival. (A) Kaplan-Meier estimates of disease-free survival (left) and overall survival (right) for a total of 56 patients with breast cancer. Marks on graph lines represent censored samples. (B) Representative images of pY36-positive and -negative breast tumor samples from the TMA cohort. (C) Kaplan-Meier estimates of disease-free survival (left) and overall survival (right) for the Stage I samples in the TMA. Marks on graph lines represent censored samples.

FIG. 17. Total ERβ IHC in breast tumors. (A) Representative images of ERβ-positive and -negative breast tumor samples from the TMA cohort. (B) Kaplan-Meier estimates of disease-free survival (left) and overall survival (right) for the Stage I samples in the TMA. Marks on graph lines represent censored samples.

FIG. 18. A model for the pY36-centered signaling circuitry.

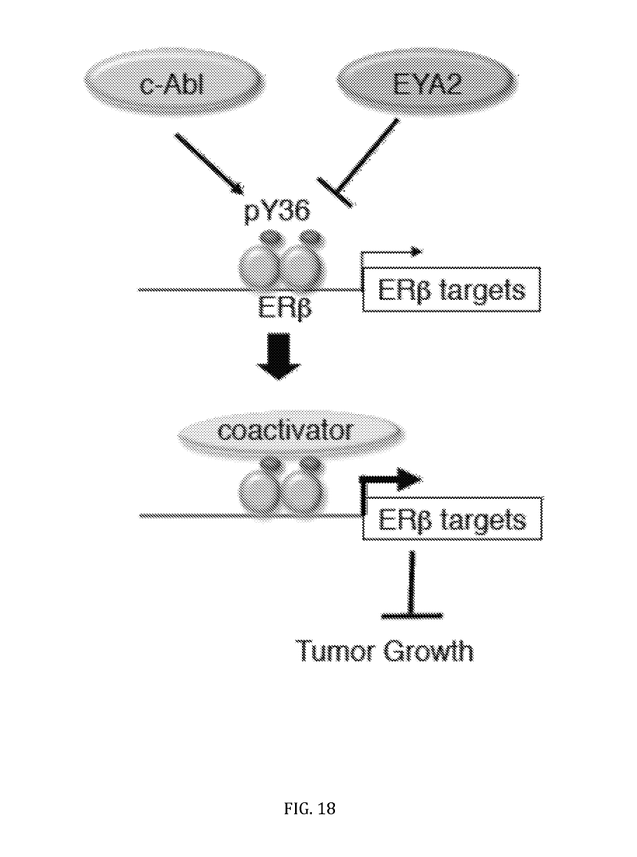

FIG. 19. Small-molecule compounds can regulate the pY36 status of ERβ. (A) C-Abl inhibitor imatinib (10 μM) reduces, while its activator DPH (30 μM) elevates, pY36 status of ectopic ERβ. E2: 10 nM. (B) An EYA2 inhibitor (10 μM) increases pY36 phosphorylation. DPN: 10 nM. (C) MDA-MB-231 cells were treated with vehicle (DMSO) or ERβ agonists (10 nM; 2 hr).

FIG. 20. Establishment of the Esr2^{Y55f/Y55F} KI model. Genotyping of the WT and KI alleles. MK: marker; ddH₂O: negative control DNA

FIG. 21. Host antitumor effect of the phosphotyrosine switch on multiple tumor types in vivo. Murine mammary tumor (A), colon tumor (B), and melanoma cells (C) were injected into syngenic WT and KI mice, and tumor volume was measured. The numbers of animals used were indicated in each graph. * p<0.05. n=8.

### DESCRIPTION

The inventors have identified an ERβ-specific phosphotyrosine residue that serves as a molecular switch for the transcriptional and antitumor activities of ERβ. Without being held to a particular theory, the inventors contemplate that c-Abl and EYA2 form a signaling circuitry together with pY36 of ERβ. As the consequence of the antagonistic actions of c-Abl and EYA2, the pY36 status dictates the functional interaction between ERβ and its coactivators. This in turn leads to transcriptional activation of ERβ-specific target genes and inhibition of tumor cell growth. This model is based on compelling data from mechanistic work in vitro, and is further bolstered by strong in vivo evidence from tumor growth studies. In particular, the fact that the Y36E mutant retains the ERβ function but bypasses the control by c-Abl and EYA2 establishes pY36 as linking ERβ with its upstream regulators. There was a stronger association of pY36-ERβ positivity with a good clinical outcome compared to total ERβ, further indicating the clinical relevance of this specific pY36-centered signaling circuitry and its potential as a therapeutic target.

The opposing actions of EYA2 and c-Abl on the pY36 status of ERβ can at least partly account for their reported activities in breast cancer. EYA2 has been shown to promote growth and invasion of breast cancer cells (Pandey et al., Oncogene, 2010, 29(25):3715-22), whereas c-Abl is reported to have a tumor-suppressive activity, at least under certain contexts (Noren et al., Nature Cell Biol., 2006, 8(8):815-25; Allington et al., FASEB J., 2009, 23(12):4231-43). However, c-Abl knockdown still increased tumor cell growth to some extent even in the presence of the constitutively active ERβ mutant, suggesting that c-Abl most likely has additional functionally important targets besides ERβ in breast cancer cells. These findings are consistent with a complex and multifaceted role of c-Abl in solid tumors (Ganguly and Plattner, Genes & Cancer, 2012, 3(5-6):414-25; Allington and Schiemann, Cells, Tissues, Organs, 2011, 193(1-2):98-113). Likewise, EYA2 may also have other substrates in addition to ERβ in promotion of breast cancer progression. Nevertheless, the data indicate that both c-Abl and EYA2 exert their opposing actions on the antitumor activity of ERβ primarily through Y36.

While glutamate (E) is generally considered as an effective phosphomimetic substitution for serine or threonine, E is structurally distinct from Y. Thus in many cases the Y-to-E mutation has the same effect on protein function as Y-to-A or Y-to-F mutations. In this regard, it is somewhat surprising that the Y36E mutant of ERβ fully retains its transcriptional and antitumor activities. This unusual property of the Y36E mutant allowed the inventors to validate the specific functional relationship between pY36 and its upstream regulators c-Abl and EYA2. Given the size difference between pY and E, it is unlikely that pY36 is directly involved in ERβ interaction with its coactivators. Rather, the negative charge at this position likely induces a conformational change in ERβ that in turn facilitates coactivator binding to other parts of ERβ.

Historically, the antitumor activity of ERβ has not been extensively exploited for breast cancer treatment. In addition, uncertainty over the clinical significance of the abundance of total ERβ further complicates efforts to develop ERβ-related agents for clinical use. As described herein, the present study of two independent clinical cohorts indicates a significant correlation between tumor pY36 status and patient survival, which substantially strengthens the clinical relevance of mechanism-based findings. Notably, pY36 status correlated with survival in Stage II and III disease, whereas no correlation was seen in Stage I disease, consistent with its predicted effect on disease progression. In support of the functional importance of pY36 for the antitumor activity of ERβ, it was found that its intensity was a more robust prognostic marker than total ERβ.

Given the druggable nature of all three components in the newly discovered signaling pathway (ERβ, EYA2, and c-Abl), the findings described herein inform the development of new approaches for breast cancer therapies. Of note, approximately half of triple negative breast cancer cases express ERβ (Marotti et al., Modern Pathology, 2010, 23(2):197-204), making stimulation of the ERβ antitumor activity an attractive therapeutic possibility for this aggressive subtype of breast cancer that currently lacks any target therapies. Indeed, there have been increasing interests in treating breast cancer and other ERβ-expressing cancers with ERβ-specific agonists (Gallo et al., Curr Pharm Des., 2012, 18(19):2734-57). The safety and drug tolerance of at least one ERβ agonist, S-equol, has been demonstrated by two completed and published clinical trials (Jackson et al., Nutrition Rev., 2011, 69(8):432-48; Ishiwata et al., Menopause, 2009, 16(1):141-8). Furthermore, small-molecule EYA2 inhibitors and c-Abl activators are available for preclinical studies (Yang et al., Chem & Biol., 2011, 18(2):177-86; Krueger et al., J Biomol Screen, 2012, (18)85-96). Given the oncogenic property of EYA2 and the context-dependent antitumor activity of c-Abl in breast cancer literature (Zhang et al., Cancer Res., 2005, 65(3):925-32; Pandey et al., Oncogene, 2010′ 29(25):3715-22; Farabaugh et al., Oncogene, 2012, 31(5):552-62; Noren et al., Nature Cell Biol. 2006, 8(8):815-25; Allington et al., FASEB J., 2009, 23(12):4231-43), it is conceivable that ERβ agonists may synergize with c-Abl activators and/or EYA2 inhibitors in inhibiting those breast tumors where the pY36-centered signaling circuitry is functional.

### I. BREAST CANCER

Breast cancer is the second most common cancer among women in the United States, second only to skin cancer. A woman in the U.S. has a one in eight chance of developing breast cancer during her lifetime. Breast cancer is the second leading cause of cancer deaths in women, with more than 40,000 deaths annually. Improved detection methods, mass screening, and advances in treatment have significantly improved the outlook for woman diagnosed with breast cancer. Approximately 80% of breast cancer cases are diagnosed in the early stages of the disease when survival rates are at their highest. As a result, about 85% percent of breast cancer patients are alive at least five years after diagnosis. Despite these advances, approximately 20% of women diagnosed with early-stage breast cancer have a poor ten-year outcome and will suffer disease recurrence, metastasis or death within this time period.

Significant research has focused on identifying methods and factors for assessing breast cancer prognosis and predicting therapeutic response. (See generally, Ross and Hortobagyi, eds. (2005) Molecular Oncology of Breast Cancer (Jones and Bartlett Publishers, Boston, Mass.) and the references cited therein). Prognostic indicators include conventional factors, such as tumor size, nodal status, and histological grade, as well as molecular markers that provide some information regarding prognosis and likely response to particular treatments. For example, determination of estrogen (ER) and progesterone (PR) steroid hormone receptor status has become a routine procedure in assessment of breast cancer patients. See, for example, Fitzgibbons et al., Arch. Pathol. Lab. Med., 2000, 124:966-78. Tumors that are hormone receptor positive are more likely to respond to hormone therapy and also typically grow less aggressively, thereby resulting in a better prognosis for patients with ER+/PR+ tumors. Overexpression of human epidermal growth factor receptor 2 (HER-2/neu), a transmembrane tyrosine kinase receptor protein, has been correlated with poor breast cancer prognosis (see, e.g., Ross et al., The Oncologist, 2003, 8:307-25), and Her-2 expression levels in breast tumors are used to predict response to the anti-Her-2 monoclonal antibody therapeutic trastuzumab (Herceptin®, Genentech, South San Francisco, Calif.).

As described herein, a number of clinical and prognostic breast cancer factors are known in the art and are used to predict treatment outcome and the likelihood of disease recurrence. Such factors include, for example, lymph node involvement, tumor size, histologic grade, family history, estrogen and progesterone hormone receptor status, Her-2 levels, and tumor ploidy. As used herein, estrogen and progesterone hormone receptor status refers to whether these receptors are expressed in the breast tumor of a particular breast cancer patient. Thus, an “estrogen receptor-positive patient” displays ER expression in a breast tumor, whereas an “estrogen receptor-negative patient” does not. Using the methods of the present invention, the prognosis of a breast cancer patient can be determined independent of or in combination with assessment of these or other clinical and prognostic factors. In some embodiments, combining the methods disclosed herein with evaluation of other prognostic factors may permit a more accurate determination of breast cancer prognosis. The methods of the invention may be coupled with analysis of, for example, Her-2 expression levels. Other factors, such as patient clinical history, family history and menopausal status, may also be considered when evaluating breast cancer prognosis via the methods of the invention. In some embodiments, patient data obtained via the methods disclosed herein may be coupled with analysis of clinical information and existing tests for breast cancer prognosis to develop a reference laboratory prognostic algorithm. Such algorithms find used in stratifying breast cancer patients, particularly early-stage breast cancer patients, into good and poor prognosis populations. Patients assessed as having a poor prognosis may be upstaged for more aggressive breast cancer treatment.

Breast cancer is managed by several alternative strategies that may include, for example, surgery, radiation therapy, hormone therapy, chemotherapy, or some combination thereof. As is known in the art, treatment decisions for individual breast cancer patients can be based on endocrine responsiveness of the tumor, menopausal status of the patient, the location and number of patient lymph nodes involved, estrogen and progesterone receptor status of the tumor, size of the primary tumor, patient age, and stage of the disease at diagnosis. Analysis of a variety of clinical factors and clinical trials has led to the development of recommendations and treatment guidelines for early-stage breast cancer by the International Consensus Panel of the St. Gallen Conference (2005). See, Goldhirsch et al., Annals Oncol., 2005, 16:1569-83. The guidelines recommend that patients be offered chemotherapy for endocrine non-responsive disease; endocrine therapy as the primary therapy for endocrine responsive disease, adding chemotherapy for some intermediate- and all high-risk groups in this category; and both chemotherapy and endocrine therapy for all patients in the uncertain endocrine response category except those in the low-risk group. Stratification of patients into poor prognosis or good prognosis risk groups at the time of diagnosis using the methods disclosed herein provides an additional or alternative treatment decision-making factor. The methods of the invention permit the differentiation of breast cancer patients with a good prognosis from those more likely to suffer a recurrence (i.e., patients who might need or benefit from additional aggressive treatment at the time of diagnosis).

The methods of the invention find particular use in choosing appropriate treatment for early-stage breast cancer patients. The majority of breast cancer patients diagnosed at an early-stage of the disease enjoy long-term survival following surgery and/or radiation therapy without further adjuvant therapy. However, a significant percentage (approximately 20%) of these patients will suffer disease recurrence or death, leading to clinical recommendations that some or all early-stage breast cancer patients should receive adjuvant therapy (e.g., chemotherapy). The methods of the present invention find use in identifying this high-risk, poor prognosis population of early-stage breast cancer patients and thereby determining which patients would benefit from continued and/or more aggressive therapy and close monitoring following treatment. For example, early-stage breast cancer patients assessed as having a poor prognosis by the methods disclosed herein may be selected for more aggressive adjuvant therapy, such as chemotherapy, following surgery and/or radiation treatment. In particular embodiments, the methods of the present invention may be used in conjunction with the treatment guidelines established by the St. Gallen Conference to permit physicians to make more informed breast cancer treatment decisions.

The present methods for evaluating breast cancer prognosis can also be combined with other prognostic methods (e.g., assessment of conventional clinical factors, such as tumor size, tumor grade, lymph node status, and family history) additional molecular markers known in the art (e.g., estrogen and progesterone hormone receptors, Her-2 and p53) and additional microarrays (e.g., Agilent (van′t Veer et al., N. Engl. J. Med., 2002, 347:1999-2009) and Affymetrix (Pawitan et al., Cancer Res., 2005, 7:953-64) for purposes of selecting an appropriate breast cancer treatment. By “microarray” is intended an ordered arrangement of hybridizable array elements, such as, for example, polynucleotide probes, on a substrate.

The methods disclosed herein also find use in predicting the response of a breast cancer patient to a selected treatment. By “predicting the response of a breast cancer patient to a selected treatment” is intended assessing the likelihood that a patient will experience a positive or negative outcome with a particular treatment. As used herein, “indicative of a positive treatment outcome” refers to an increased likelihood that the patient will experience beneficial results from the selected treatment (e.g., complete or partial remission, reduced tumor size, etc.). By “indicative of a negative treatment outcome” is intended an increased likelihood that the patient will not benefit from the selected treatment with respect to the progression of the underlying breast cancer. In some aspects of the invention, the selected treatment is chemotherapy, c-abl, or EYa2 directed therapies. In other aspects of the invention, the selected treatment is anti-VEGF therapy, such as, for example, monoclonal antibody therapy (e.g., bevacizumab). In still other aspects of the invention, the selected treatment is anti-HIF1α therapy, such as, for example, treatment with small molecule inhibitors of HIF1α activity (see, e.g., Powis and Kirkpatrick, Mol. Cancer. Therap., 2004, 3:647-54).

In certain embodiments, methods for predicting the likelihood of survival of a breast cancer patient are provided. In particular, the methods may be used predict the likelihood of long-term, disease-free survival. By “predicting the likelihood of survival of a breast cancer patient” is intended assessing the risk that a patient will die as a result of the underlying breast cancer. “Long-term, disease-free survival” is intended to mean that the patient does not die from or suffer a recurrence of the underlying breast cancer within a period of at least five years, such as at least ten or more years, following initial diagnosis or treatment. Such methods for predicting the likelihood of survival of a breast cancer patient include detecting the presence or phosphorylation status or level of ERβ, particularly at Y36, in a sample from the patient, where low levels of pY36 is indicative of a poor likelihood of survival. Likelihood of survival can be assessed in comparison to, for example, breast cancer survival statistics available in the art.

### II. KITS

Kits for practicing the methods described are further provided. By “kit” it is intended any manufacture (e.g., a package or a container) including at least one reagent, such as an antibody or the like, for specifically detecting the phosphorylation level or status of ERβ-Y36. The kits can be promoted, distributed or sold as units for performing the methods of the present invention. Additionally, kits can contain a package insert describing the kit and methods for its use.

In particular embodiments, kits for diagnosing and for evaluating the prognosis of a breast cancer patient including detecting the phosphorylation level or status of ERβ-Y36 are provided. Such kits are compatible with both manual and automated detection techniques. These kits include, for example, at least one antibody that specifically binds phosphorylated ERβ-Y36.

In other embodiments, kits for practicing the immunohistochemistry methods of the invention are provided. Such kits are compatible with both manual and automated immunohistochemistry techniques (e.g., cell staining). These kits include at least one ERβ-Y36 antibody for specifically detecting the pY36. An antibody can be provided in the kit as an individual reagent.

Any or all of the kit reagents can be provided within containers that protect them from the external environment, such as in sealed containers. Positive and/or negative controls can be included in the kits to validate the activity and correct usage of reagents employed in accordance with the invention. Controls can include samples, such as tissue sections, cells fixed on glass slides, protein preparations from tissues or cell lines, and the like. The design and use of controls is standard and well within the routine capabilities of those of ordinary skill in the art.

### III. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Mobilizing the Antitumor Activity of Estrogen Receptor B Through a Phosphotyrosine Switch

A. Results

EYA2 Modulates Transcriptional Activity of ERβ, not ERα.

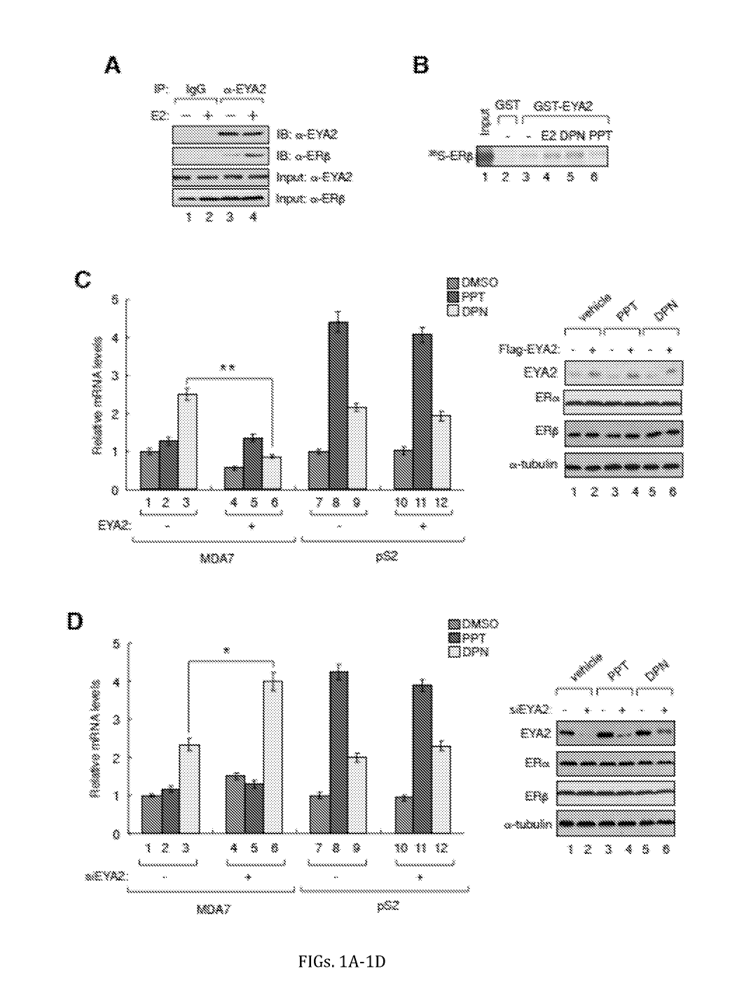

To identify proteins that specifically regulate ERβ but not ERα, ERβ AF1 (amino acid 1-148) was used, the region most divergent from ERα, as the bait in a yeast two-hybrid screen. EYA2 was isolated from the initial screen, and verified its association with ERβ by coimmunoprecipitation (co-IP) of endogenous EYA2 and ERβ in MCF7 breast cancer cells (FIG. 1A) and glutathione-S-transferase pull-down of recombinant proteins (FIG. 1B). The EYA2-ERβ interaction was detectable without any ERβ ligands, but was enhanced by the ERα/ERβ common ligand 17-β-estradiol (E2; FIG. 1A) and the ERβ-specific ligand diarylpropionitrile (DPN; FIG. 1B). In contrast, the ERα-specific ligand propyl-pyrazole triol (PPT) did not have any effects on ERβ binding to EYA2 (FIG. 1B).

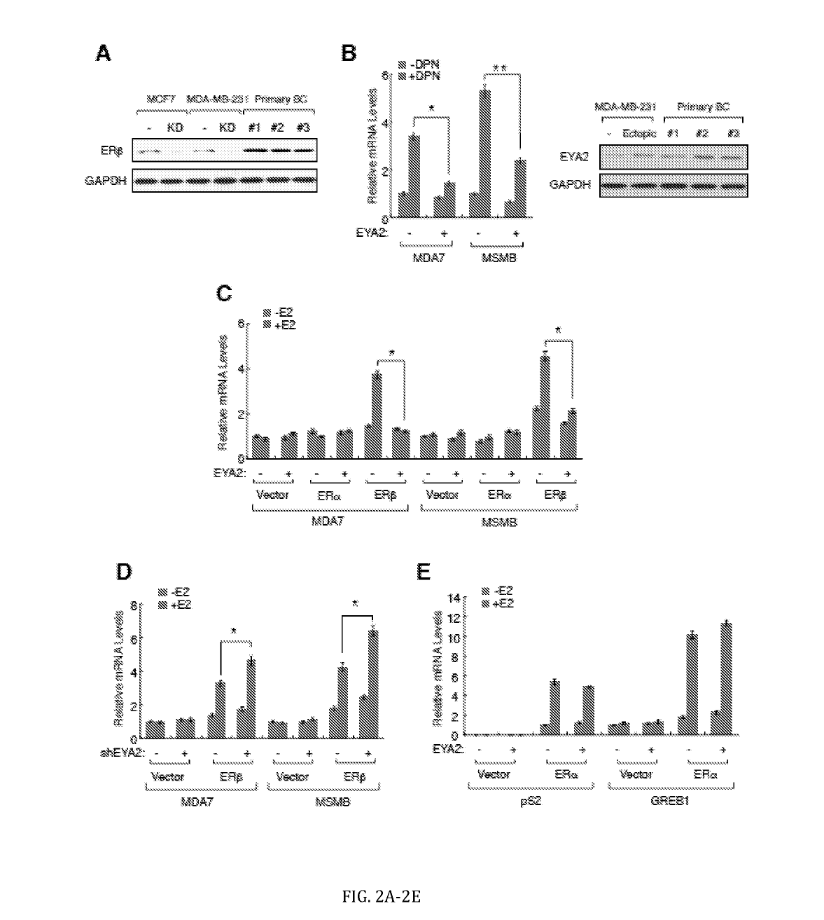

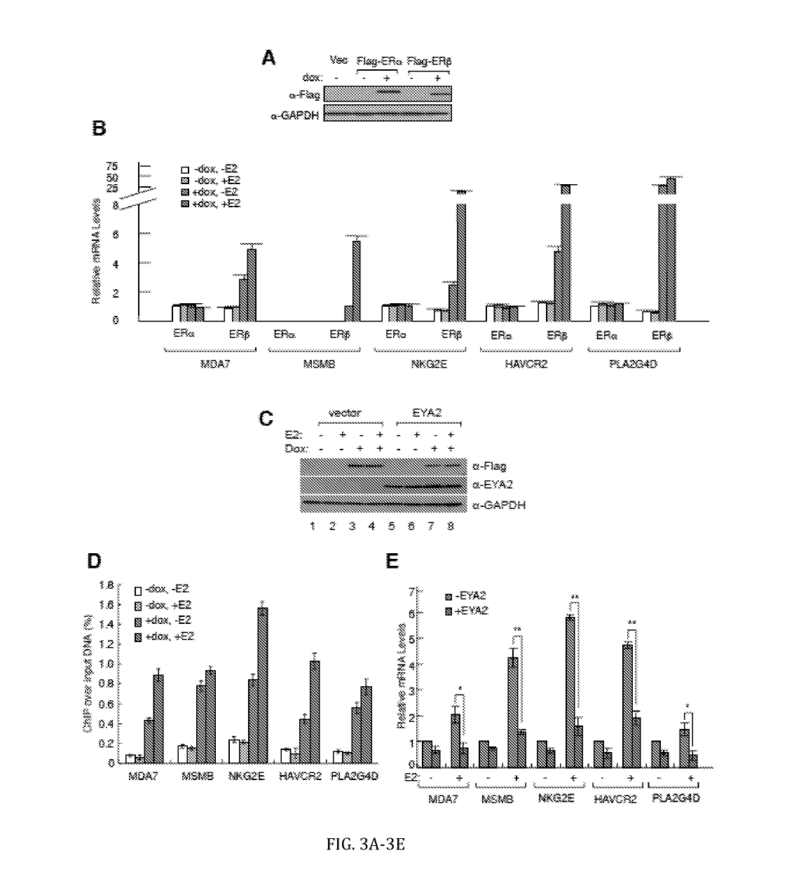

The effect of EYA2 on the transcriptional activity of ERβ was examined. MCF7 breast cancer cells express both ERα and ERβ (FIG. 2). Therefore, both ERα-specific ligand PPT and ERβ-specific ligand DPN stimulated transcription of pS2, a common target gene of ERα and ERβ (FIG. 1C, column 8-9). In contrast, only DPN but not PPT activated transcription of MDA7, an ERβ-specific target (FIG. 1C, column 2-3). Ectopic expression of EYA2 repressed transcriptional activation of MDA7 by DPN (FIG. 1C, compare column 3 and 6), but not that of pS2 by either DPN or PPT (FIG. 1C, compare column 7-9 with 10-12). Reciprocally, siRNA knockdown of EYA2 further enhanced transcriptional activation of MDA7 by DPN (FIG. 1D; compare column 3 and 6), without affecting DPN or PPT-activated transcription of pS2 (FIG. 1D, compare column 7-9 with 10-12). A similar repressive effect of EYA2 on ERβ-mediated transcription was also observed in MDA-MB-231 breast cancer cells, which express ERβ but not ERα (FIGS. 2, A and B). Furthermore, ERα or ERβ were introduced into HEK293T and breast cancer Hs578T cell line, both of which lack endogenous ERα/β expression. It was confirmed that the ERβ-specific transcriptional repression by EYA2 on multiple ERβ-specific target genes in the ERα/β-reconstituted HEK293T (FIGS. 2, C, D, and E) and Hs578T cells (FIG. 3). Taken together, these data strongly suggest that EYA2 is a transcriptional corepressor of ERβ, but not ERα.

EYA2 Inhibits ERβ Transcriptional Activity by Directly Dephosphorylating pY36 of ERβ.

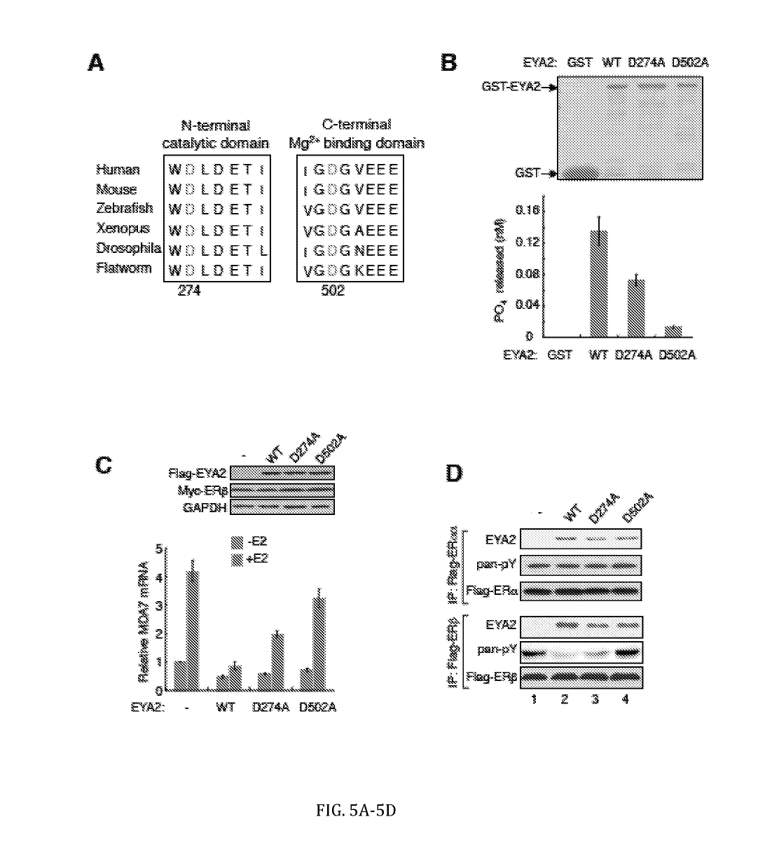

To understand how EYA2 repressed the activity of ERβ but not ERα, it was first determined whether the tyrosine phosphatase activity of EYA2 was required for its transcriptional repression. Two point mutations of EYA2 were engineered that either partially (D274A) or completely (D502A) eliminated its phosphatase activity (FIGS. 5, A and B). The impaired enzymatic activity of these two EYA2 mutants correlated with the degree of their deficiency in repressing ERβ-mediated transcriptional activation (FIG. 5C). Furthermore, wild type (WT) EYA2 significantly diminished the total tyrosine phosphorylation (pY) level of ERβ, whereas the two EYA2 mutants were deficient in reducing pY of ERβ (FIG. 5D). In contrast, WT-EYA2 did not affect the total pY status of ERα (FIG. 5D). These results are consistent with the notion that EYA2 represses the transcriptional activity of ERβ by directly dephosphorylating a certain phosphotyrosine residue in ERβ.

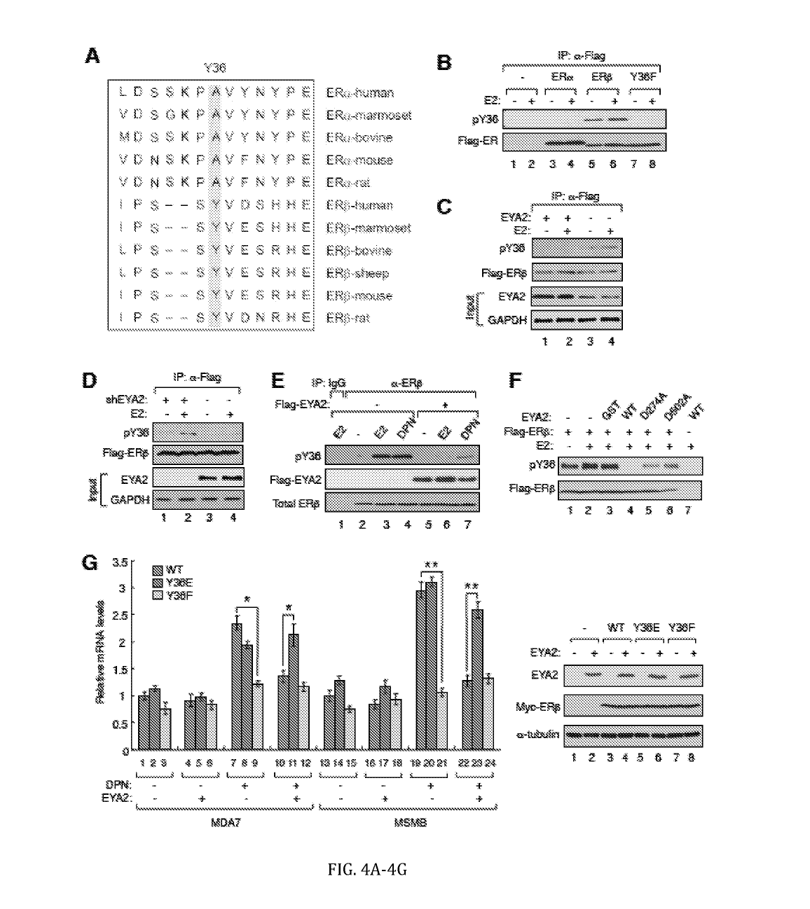

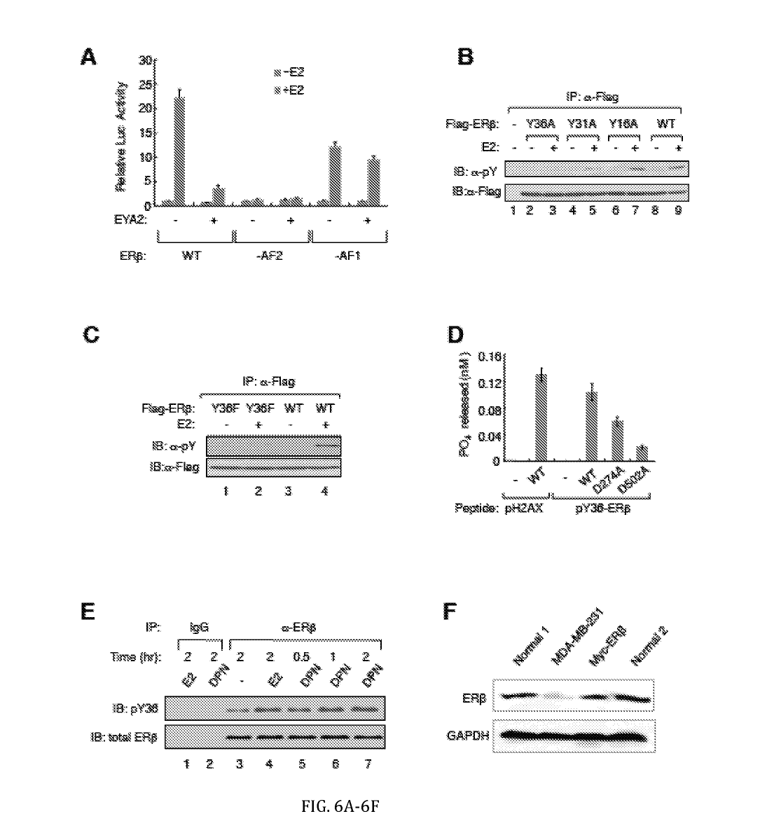

To identify the EYA2-targeted phosphotyrosine residue in ERβ, the inventors focused on the AF1 domain of ERβ because of its sequence divergence from ERα. Indeed, an ERβ mutant lacking AF1, while still retaining partial ligand-dependent transcription activity, was refractory to EYA2-mediated repression (FIG. 6A). By systematically mutating individual tyrosine residues in AF1 of ERβ, it was found that substitution of Y36 with either alanine (Y36A) or phenylalanine (Y36F) largely abolished the total pY signal of ERβ (FIGS. 6B and 6C). Y36 is highly conserved among ERβ orthologs in mammals, but interestingly, human and other mammalian ERα proteins have an alanine residue at the corresponding position (FIG. 4A). A pY36-containing ERβ peptide that can be dephosphorylated efficiently by recombinant WT-EYA2 (FIG. 6D). This phospho-peptide was used as the antigen to raise a phospho-specific polyclonal antibody that recognized WT-ERβ, but not Y36F-ERβ or WT-ERα (FIG. 4B). The pY36 signal was substantially reduced by EYA2 overexpression (FIG. 4C) and enhanced by EYA2 knockdown (FIG. 4D), thus further validating the antibody specificity. Furthermore, pY36 of endogenous ERβ in MDA-MB-231 and MCF7 breast cancer cells was stimulated by ERβ agonists (FIG. 4E and FIG. 6E), and dampened by EYA2 (FIG. 4E). Lastly, recombinant WT, but not mutant EYA2 completely eliminated the pY36 signal of ERβ in vitro (FIG. 4F). Collectively, these results unequivocally demonstrate that pY36 of ERβ is a direct substrate of the EYA2 tyrosine phosphatase activity.

To examine the impact of pY36 on ERβ-mediated transcription, we expressed WT and mutant ERβ in MDA-MB-231 breast cancer cells at levels comparable to that observed for endogenous ERβ in normal breast tissue (FIG. 6F). The Y36F mutation abolished the ligand-dependent activation of the ERβ target genes (FIG. 4G, compare column 7 with 9, and 19 with 21). In contrast, a tyrosine-to-glutamate (Y36E) mutation retained the transcriptional activity of ERβ (FIG. 4G, compare column 7 with 8, and 19 with 20), suggesting that a negative charge at this position was sufficient to sustain ERβ transcriptional activity. Remarkably, unlike WT-ERβ, Y36E-ERβ was largely refractory to EYA2-mediated transcriptional repression (FIG. 4G, compare column 10 with 11, and 22 with 23). This finding lends strong support to the notion that EYA2 represses the transcriptional activity of ERβ primarily through dephosphorylation of pY36. As ERα lacks a tyrosine residue at the corresponding position and its overall pY intensity is not affected by EYA2 (FIG. 5D), the data provide a molecular explanation for the repressive effect of EYA2 on transcriptional activity of ERβ, not ERα.

c-Abl Directly Phosphorylates Y36 and Promotes ERβ-Mediated Transcriptional Activation.

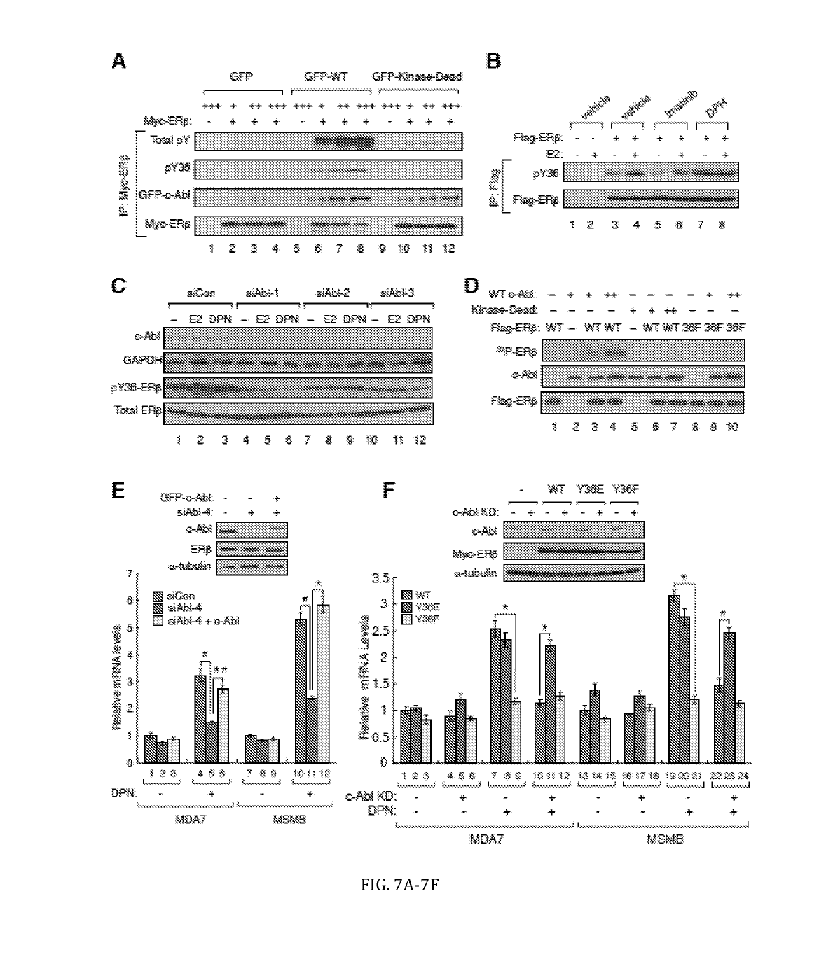

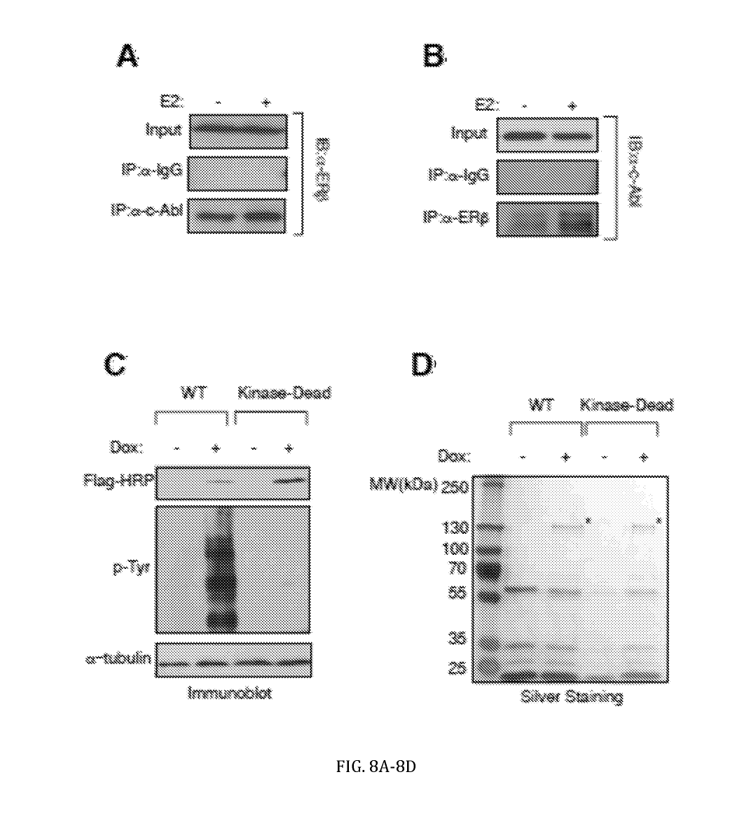

In order to identify the tyrosine kinase that phosphorylates Y36, a mammalian expression library was screened that contains all known human tyrosine kinases. The initial screen identified c-Abl as a candidate kinase for Y36 phosphorylation. Follow-up experiments as described below confirm that c-Abl directly phosphorylates Y36. First, WT c-Abl, but not a kinase-dead mutant (Tsai and Yuan, Cancer Res., 2003, 63(12):3418-24), significantly increased both the total pY and pY36 levels of ERβ (FIG. 7A). Second, both ectopic and endogenous c-Abl and ERβ were physically associated with each other in co-IP (FIG. 7A; FIGS. 8A and 8B). Third, the c-Abl inhibitor imatinib reduced the pY36 intensity (FIG. 7B, compare lane 3-4 with 5-6), whereas a c-Abl activator DPH (34) stimulated Y36 phosphorylation (FIG. 7B, compare lane 3-4 with 7-8). Fourth, knockdown of c-Abl by multiple independent siRNA oligonucleotides dampened Y36 phosphorylation (FIG. 7C). Lastly, affinity-purified WT c-Abl protein, but not the kinase-dead mutant (Stuart et al., Oncogene, 2005, 24(55):8085-92), directly phosphorylated ERβ in an in vitro kinase reaction (FIG. 7D, compare lane 3-4 with 6-7; FIGS. 8, C and D). In contrast, the Y36F mutant of ERβ was not phosphorylated by WT c-Abl (FIG. 7D, lane 9 and 10). In aggregate, these data clearly demonstrate that Y36 is the primary substrate for the c-Abl kinase activity.

To assess the functional impact of c-Abl on the transcriptional activity of ERβ, the effect of c-Abl knockdown on ligand-stimulated transcription of the ERβ target genes was analyzed. To ascertain specificity of the siRNA knockdown, MDA-MB-231 cells were transfected with an siRNA oligonucleotide that targets the 3′ untranslated region (3′-UTR) of the c-Abl gene, and for the rescuing purpose, used a WT c-Abl expression vector lacking the corresponding 3′-UTR sequence. The c-Abl knockdown alone significantly reduced the DPN-stimulated mRNA levels of MDA7 and MSMB, two ERβ target genes (FIG. 7E, compare lane 4 with 5, and 10 with 11). Co-transfection of the siRNA-resistant cDNA clone of c-Abl rescued the knockdown effect (FIG. 7E, lane 6 and 12), thus validating the role of c-Abl in supporting ERβ-dependent transcription.

To determine whether the c-Abl effect on ERβ-dependent transcription was through phosphorylation of Y36, the effect of c-Abl knockdown on WT-ERβ, Y36F, and Y36E mutants were compared. While Y36F remained transcriptionally inactive upon c-Abl knockdown (FIG. 7F, compare lane 9 with 12, and 21 with 24), the transcriptionally active yet nonphosphorylatable Y36E mutant was refractory to c-Abl knockdown (FIG. 7F, compare lane 8 with 11, and 20 with 23). This is reminiscent of recalcitrance of the same ERβ mutant to the transcriptional repression by EYA2 (FIG. 4G). Taken together, these enzymatic and transcriptional results establish a functional relationship between c-Abl/EYA2 and their common downstream target, Y36 of ERβ.

pY36 Promotes the Interaction Between ERβ and its Coactivators.

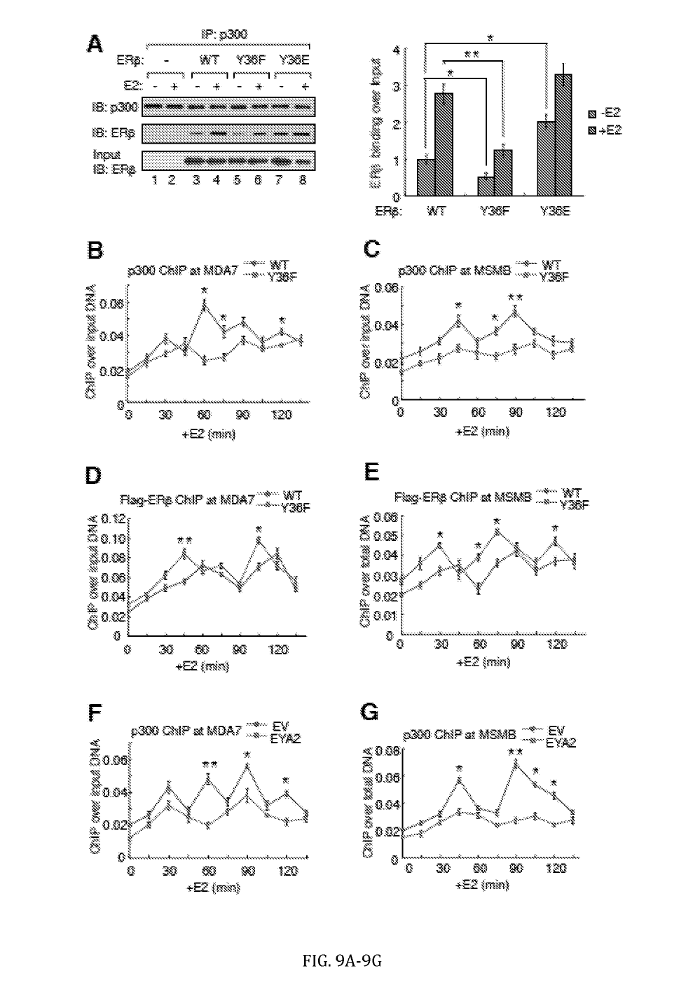

To elucidate the molecular basis for the role of pY36 in transcriptional activation, the ability of WT-ERβ and Y36 mutants to bind to p300, one of the known transcriptional coactivators of ERβ, was compared (Ohtake et al., Nature, 2003, 423(6939):545-50; Bouchal et al., The Prostate, 2011, 71(4):431-7). The Y36F mutant had a significantly reduced affinity for p300 as compared to WT-ERβ (FIG. 9A, compare lane 3-4 with 5-6). In contrast, the Y36E mutant had a somewhat high affinity for p300 than WT-ERβ (FIG. 9A, lane 7-8, also see quantification).

Next, chromatin immunoprecipitation (ChIP) was used to compare the ability of WT-ERβ and the Y36F mutant to recruit p300 to the ERβ target promoters. Consistent with published studies of ERα (Metivier et al., Cell, 2003, (115)751-63), E2 treatment stimulated cyclic recruitment of p300 to the ERβ target promoters in WT-ERβ-expressing cells (FIGS. 9B and 9C). In contrast, cells expressing the Y36F mutant exhibited substantially attenuated ligand-dependent recruitment of p300 (FIGS. 9B and 9C). This was not due to reduced chromatin binding of the Y36F mutant to these promoters (FIGS. 9D and 9E). In fact, more Y36F was associated with the promoter regions than was WT-ERβ. In a separate ChIP, it was found that EYA2 also reduced promoter recruitment of p300 (FIGS. 9F and 9G; FIG. 3C), which phenocopied the mutational effect of Y36F (FIGS. 9B and 9C). These data strongly indicate that pY36 is important for ERβ-mediated coactivator binding and promoter recruitment.

The pY36 Signaling Circuitry Regulates the Antitumor Activity of ERβ.

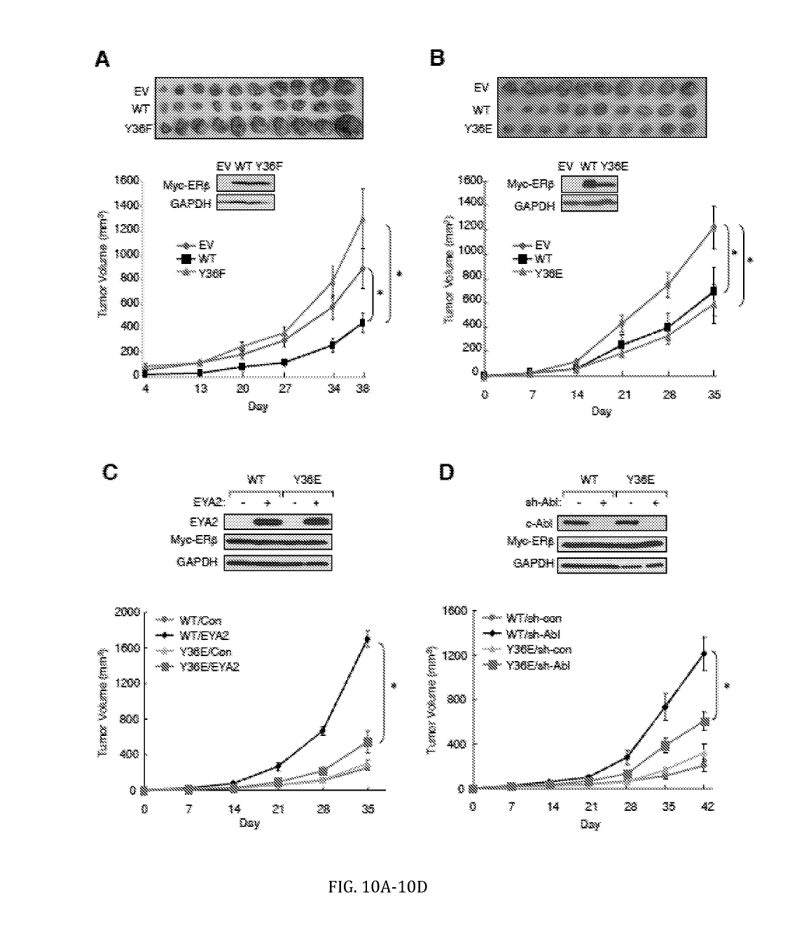

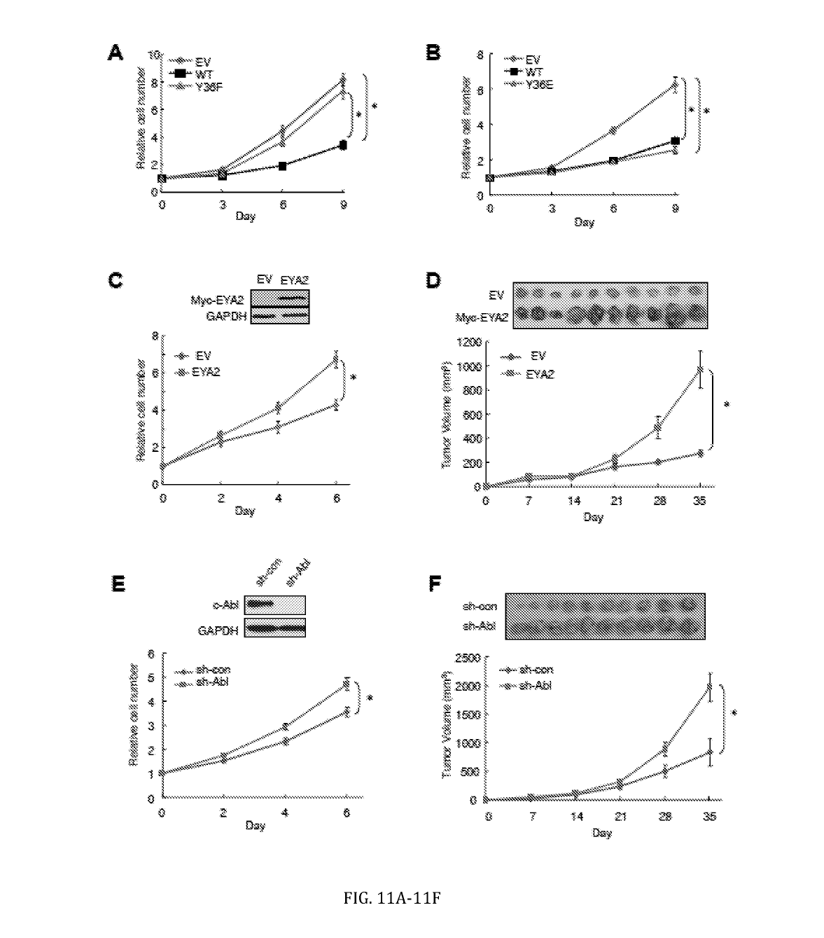

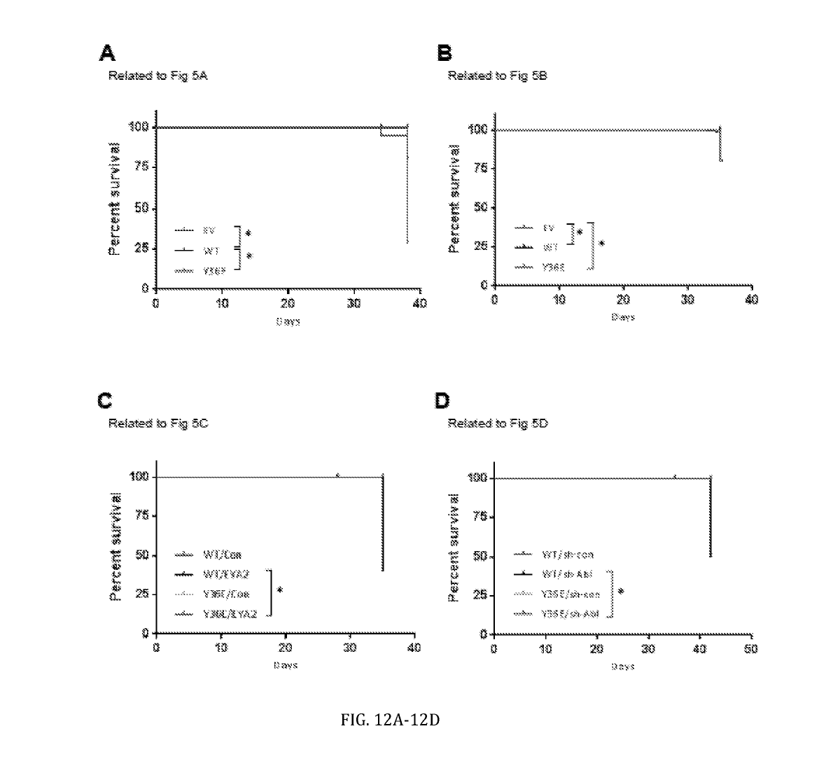

To determine the importance of the pY36 signaling circuitry in the antitumor function of ERβ, the effects of WT and mutant ERβ proteins on tumor cell growth were compared. Consistent with published work (Hartman et al., Cancer Res., 2006, 66(23):11207-13; Mak et al., Neoplasia, 2006, 8(11):896-904; Hodges-Gallagher et al., Breast Cancer Res Treat., 2008, 109(2):241-50; Thomas et al., Breast Cancer Res., 2012, 14(6):R148), WT-ERβ significantly reduced the growth of breast cancer cells in both tissue culture (FIGS. 11A and 11B) and xenograft models (FIGS. 10A and 10B; FIGS. 12A and 12B). In contrast, the transcriptionally inactivating Y36F mutation completely abolished ERβ antitumor activity (FIG. 10A; FIG. 11A). On the other hand, the transcriptionally active Y36E mutant inhibited tumor cell growth as robustly as the WT protein (FIG. 10B and FIG. 11B). These results clearly indicate that pY36 is important for the antitumor activity of ERβ.

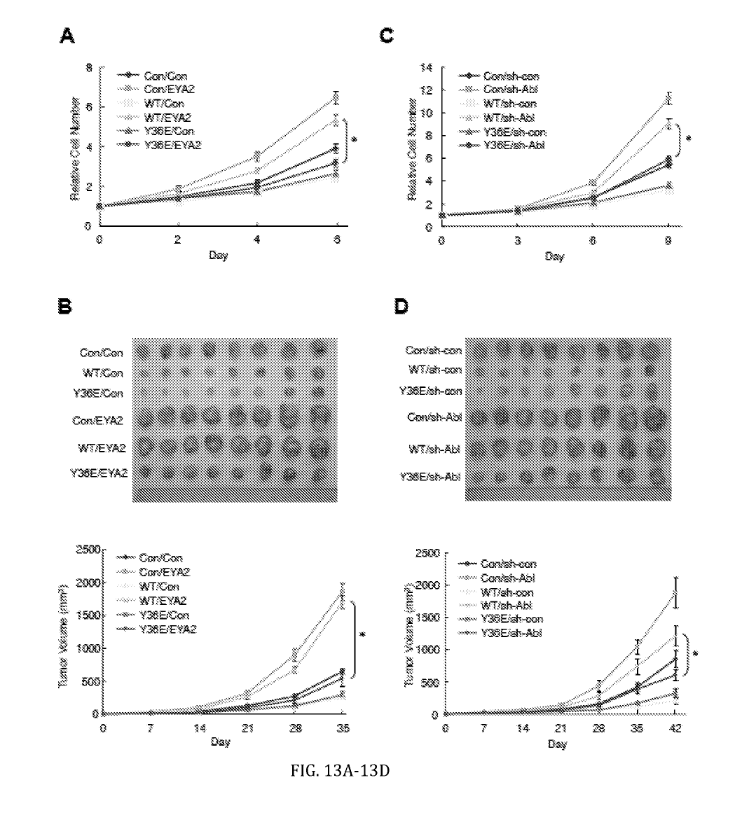

Next, the relevance of EYA2 and c-Abl to the antitumor activity of ERβ was validated. Consistent with the previous reports of oncogenic activity of EYA2 (Pandey et al., Oncogene, 2010, 29(25):3715-22; Farabaugh et al., Oncogene, 2012, 31(5):552-62) in breast cancer, ectopic expression of EYA2 promoted breast cancer cell growth in vitro and in vivo (FIGS. 11C and 11D). Using the same systems, it was found that knockdown of c-Abl led to accelerated tumor cell growth (FIGS. 11E and 11F). The inventors reasoned that, if the effects of EYA2 and c-Abl on tumor cell growth were mediated by the phosphorylation status of Y36-ERβ, they would differentially influence the antitumor activity of WT-ERβ and the functionally active yet non-phosphorylatable Y36E mutant. Indeed, both in vitro and in vivo studies showed that EYA2 substantially neutralized the antitumor activity of WT-ERβ, but not that of the Y36E mutant (FIG. 10C; FIGS. 12C; 13A and 13B). Likewise, the same Y36E mutant was relatively refractory to c-Abl knockdown as compared to WT-ERβ (FIG. 10D; FIG. 12D; FIGS. 13C and 13D). Therefore, Y36E-ERβ retains the antitumor activity of WT-ERβ but circumvents the control by c-Abl and EYA2. Based on these findings, it was concluded that EYA2 and c-Abl regulate the antitumor activity of ERβ predominantly via their influence over the phosphorylation status of the Y36 residue.

High pY36 is a Prognostic Marker for Breast Cancer Progression and Predicts Longer Survival.

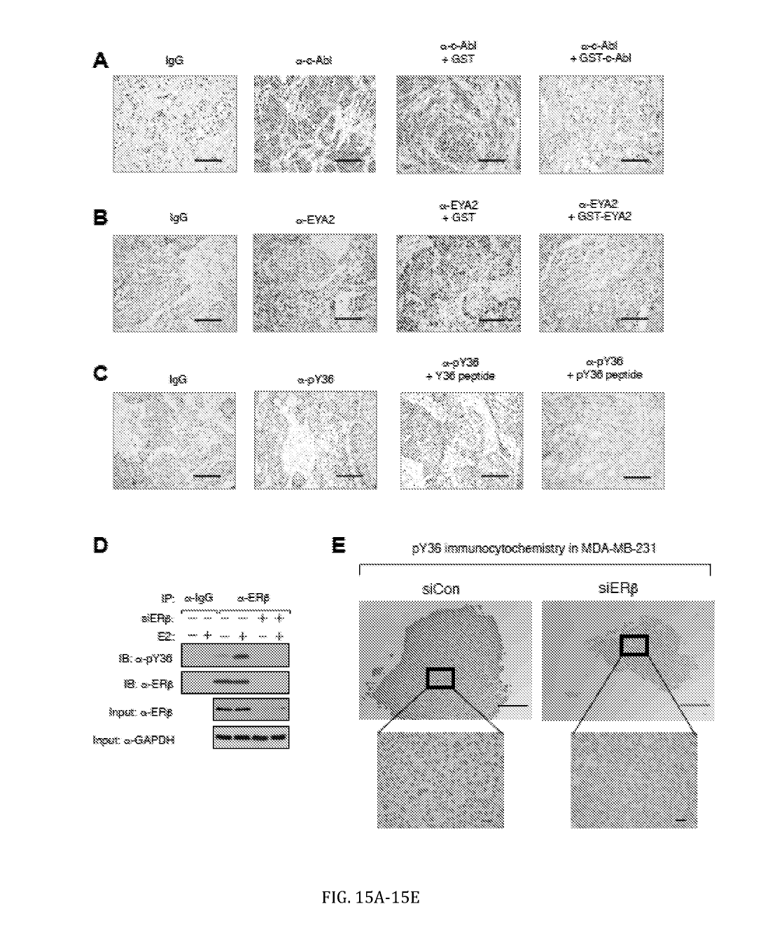

To explore the clinical significance of this newly discovered signaling circuitry, immunohistochemistry (IHC) of breast cancer tissue samples was conducted. First, the specificity of the antibodies for pY36, EYA2, and c-Abl used in IHC was verified by antigen competition (FIGS. 15A, 15B, and 15C). In particular, prominent nuclear staining of breast tumor cells with the pY36 antibody was abolished by preincubation of the antibody with a pY36-containing peptide, but not its non-phosphorylated counterpart (FIG. 15C). In addition, siRNA knockdown of endogenous ERβ in MDA-MB-231 cells abolished the pY36 signal in both IP-Western and immunocytochemistry (FIGS. 15D and 15E), thus further corroborating the specificity of this antibody.

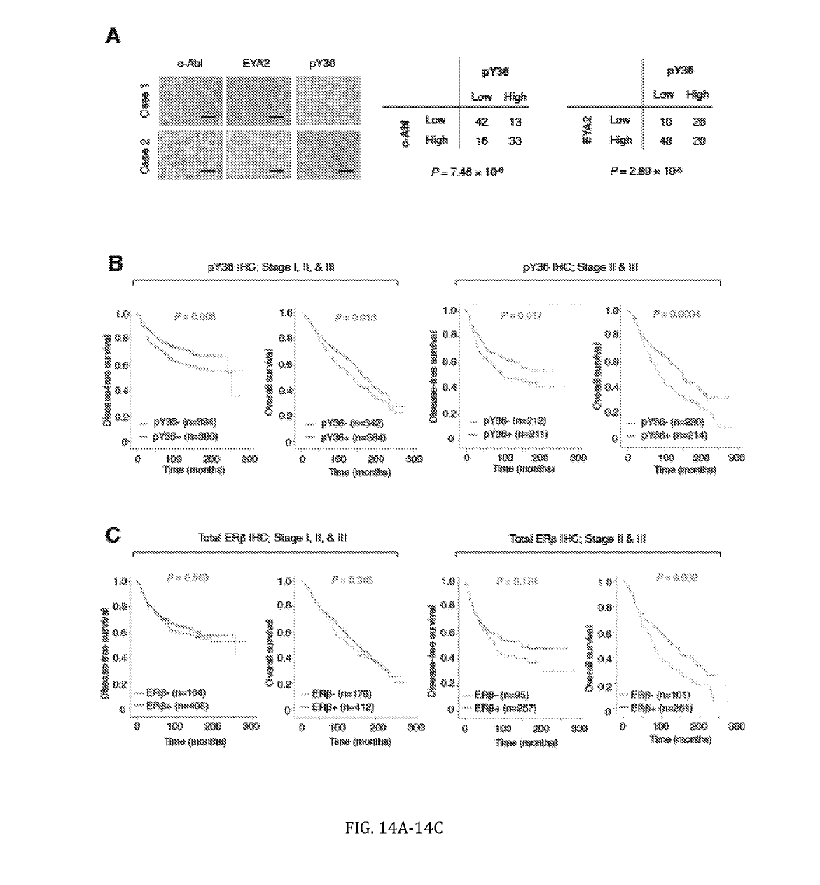

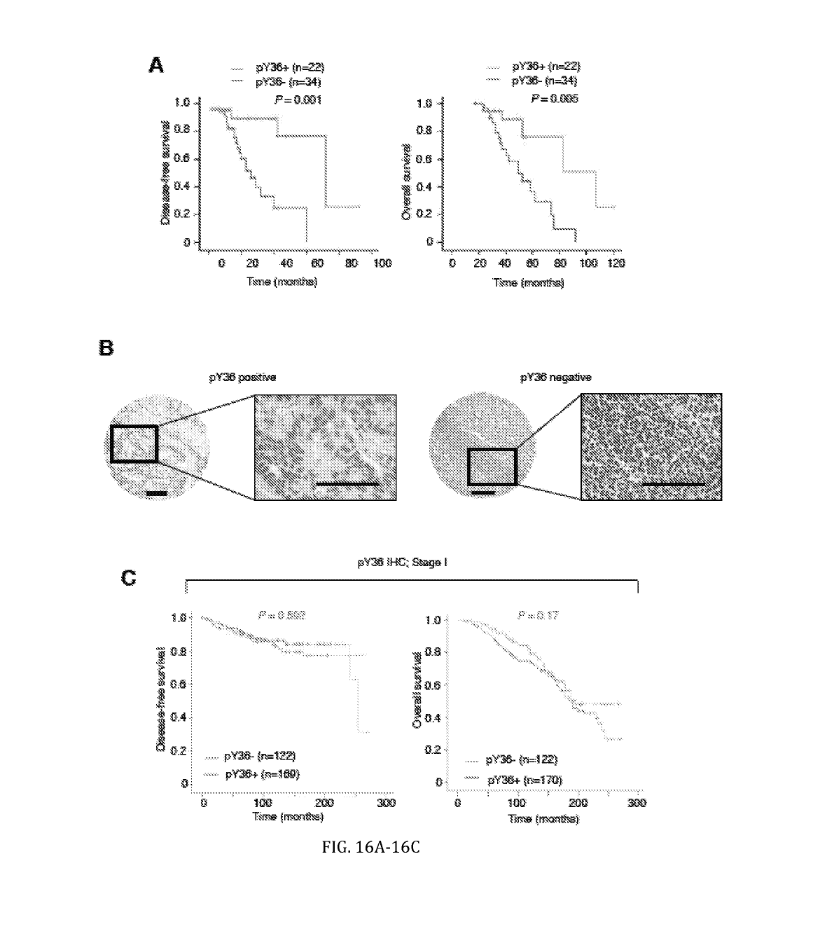

Using a cohort of 104 human breast tumor samples in the initial study, a markedly positive correlation (P=7.46×10⁻⁶) was found between c-Abl and pY36 levels, and a negative correlation (P=2.89×10⁻⁵) between EYA2 and pY36 levels (FIG. 14A). This is consistent with the opposite effects of c-Abl and EYA2 on the pY36 status and ERβ functions observed in preclinical studies. Using the available survival information of 56 subjects from this cohort, a strong correlation between positive pY36 staining and longer disease-free (P=0.001) and overall survival (P=0.005; FIG. 16A) was observed.

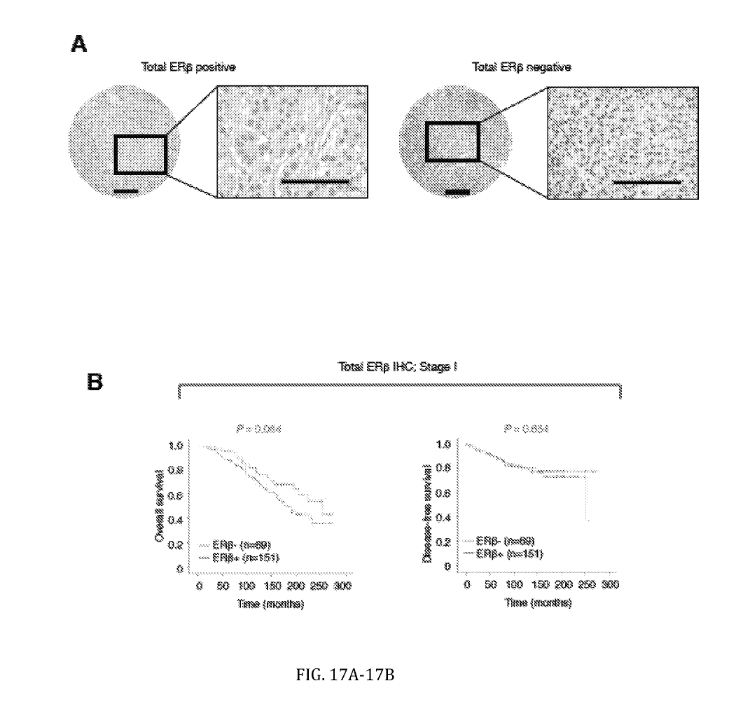

To validate the clinical correlation, a Prognostic Tissue Microarray (TMA) from the National Cancer Institute, which consists of a larger cohort of breast tumor samples with a clinical follow-up record, was used. Using a total of 726 readable IHC samples, it was found that the pY36 signal was inversely correlated with tumor size (P=1.1×10⁻⁶), positive node status (P=0.034), advanced disease stage (P=8.86×10⁻⁶), and increased tumor grade (P=0.007), thus demonstrating a significant correlation between loss of pY36 and disease progression (FIG. 16B and Table 1). Patients with pY36-negative tumors had shorter disease-free (P=0.006) and overall (P=0.013) survival than those with pY36-positive tumors (FIG. 14B, left graphs). Most strikingly, the association with survival was only seen in Stage II and III disease, consistent with an effect of pY36 on disease progression (FIG. 14B, right graphs; and FIG. 16C). As a comparison, the inventors also performed IHC of total ERβ using a previously validated commercial antibody (39) (FIG. 17A). None of the ERβ-negative samples was stained positive for pY36 (data not shown), further corroborating the specificity of the pY36. Unlike pY36, the inventors did not find statistically significant correlation between total ERβ and disease progression when all disease stages were combined (FIG. 14C, left graphs). When only Stage II and III cases were considered, total ERβ levels correlated with overall (P=0.002) but not disease-free (P=0.134) survival (FIG. 14C, right graphs; FIG. 17B). Thus, pY36 intensity appears to have a stronger and more specific correlation with disease free survival than total ERβ, underscoring the clinical relevance of the previously unappreciated, pY36-centered signaling circuitry.

TABLE 1
Correlations between pY36, ERβ status and clinical factors.

| Clinical | Total | pY36 | pY36 | P value | Total | ERβ | ERβ | P value |
|---|---|---|---|---|---|---|---|---|
| characteristics | cases | negative | positive | | cases | negative | positive | |
| | (pY36) | | | | (ERβ) | | | |
| Tumor size | 726 | | | 1.1 × 10⁻⁶ | 583 | | | 0.123 |
| ≦20 mm | 392 | 152 | 240 | | 296 | 78 | 318 | |
| >20 mm | 334 | 190 | 144 | | 286 | 92 | 194 | |
| Nodal status | 726 | | | 0.034 | 582 | | | 0.48 |
| Negative | 389 | 169 | 220 | | 294 | 83 | 212 | |
| Positive | 337 | 173 | 164 | | 288 | 88 | 200 | |
| Stage | 726 | | | 8.86 × 10⁻⁶ | 582 | | | 2.91 × 10⁻⁶ |
| I | 292 | 122 | 170 | | 220 | 69 | 151 | |
| II | 280 | 121 | 159 | | 222 | 41 | 181 | |
| III | 154 | 99 | 55 | | 140 | 60 | 80 | |
| Grade | 726 | | | 0.007 | 582 | | | 0.141 |
| I | 169 | 71 | 98 | | 136 | 46 | 91 | |
| II | 329 | 144 | 185 | | 258 | 80 | 178 | |
| III | 228 | 127 | 101 | | 188 | 45 | 143 | |
| ER | 726 | | | 1.26 × 10⁻⁶ | 582 | | | 0.012 |
| Negative | 202 | 120 | 76 | | 154 | 56 | 98 | |
| Positive | 504 | 206 | 298 | | 402 | 103 | 209 | |
| Unknown | 20 | | | | 26 | | | |
| PR | 726 | | | 4.8 × 10⁻⁶ | 583 | | | 0.009 |
| Negative | 245 | 140 | 105 | | 197 | 70 | 127 | |
| Positive | 453 | 186 | 267 | | 380 | 90 | 270 | |
| Unknown | 28 | | | | 25 | | | |
| HER2 | 726 | | | 0.308 | 582 | | | 0.238 |
| Negative | 597 | 275 | 322 | | 469 | 140 | 329 | |
| Positive | 107 | 55 | 52 | | 93 | 23 | 71 | |
| Unknown | 22 | | | | 20 | | | |

Statistical analysis
The acsociation of pY36 status with single clinical actor was mewed by Pearson chi-square test.
Statistical calculations were performed with SPSS 13.0 P-values of less than 0.05 were considered stanstically significant.

pY36 Status can be Regulated by Pharmacologic Agents.

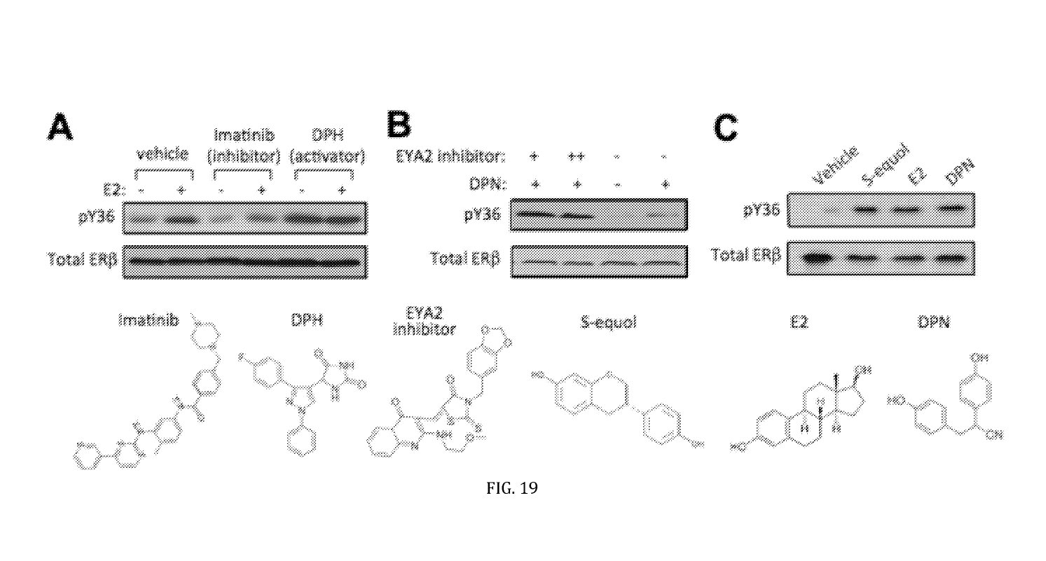

Given the druggable nature of multiple components (ERβ, EYA2, and c-Abl) in the signaling circuitry that regulates the pY36 status, the inventors provide information for the development of novel and targeted therapies. Small-molecule compounds known to target the signaling circuitry could alter the phosphorylation status of ERβ. Known c-Abl activator DPH (Yang et al. Chem & Biol, 2011, 18:177-86) (lane 5-6 in FIG. 19A) and an EYA2 inhibitor (Krueger et al. J Biomol Screen, 2012, 18:85-96) (lane 1-2 in FIG. 19B) increased, whereas the c-Abl inhibitor imatinib reduced (lane 3-4 in FIG. 19A), the ERβ phosphorylation signal. Furthermore, ERβ phosphorylation was enhanced by the ERα/ERβ agonist 17β-estradiol and two ERβ-specific agonists DPN and S-equol (FIG. 19C). These findings establish the proof of principle for targeting multiple druggable players in this signaling network.

Demonstration of the Physiological Significance of pY36 Using a Novel Mouse Knockin Model.

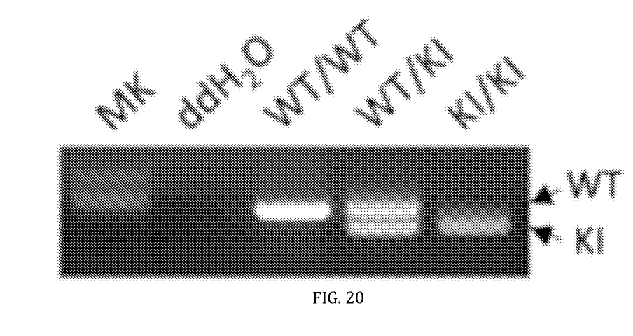

The inventors sought to ascertain the functional significance of the phosphotyrosine switch in a syngeneic animal model. To this end, a whole-body knockin (KI) mouse model (C57BL/6) was established in which a single amino acid substitution (human Y36F corresponds to mouse Y55F) was introduced into the endogenous ERβ (FIG. 20).

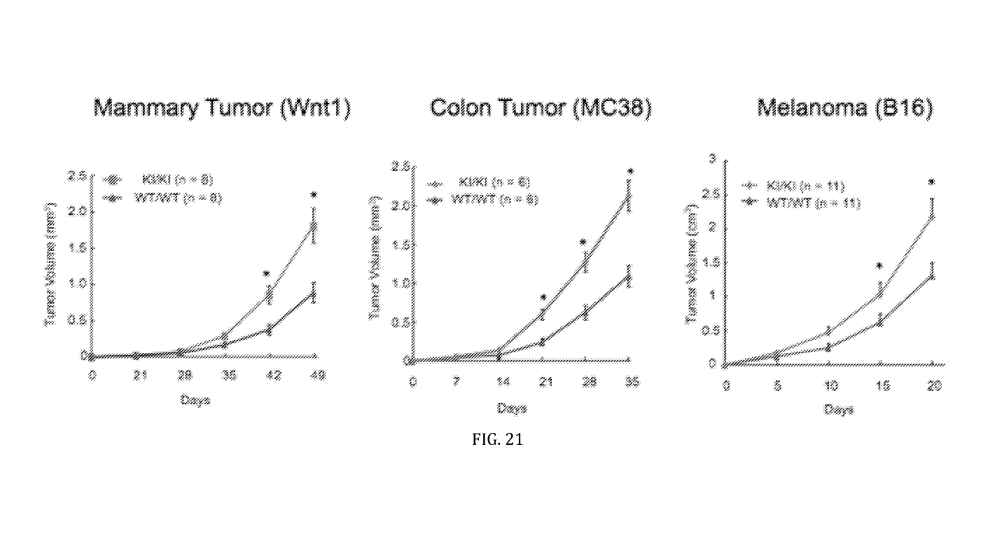

Published studies indicate that the antitumor activity of ERβ is contributed by its actions in both tumor and host cells (Fan et al. Proc Natl Acad Sci USA, 2010, 107:6453-58; Sareddy et al. Mol Cancer Ther 2012, 11:1174-82; Mak et al., Neoplasia 2006, 8:896-904; Nanni et al., J Clin Invest 2009, 119:1093-108; Mak et al., Cancer Cell 2010, 17:319-32; Nakajima et al., Sci Signal 2011, 4:ra22; Leung and Ho, Sci Signal 2011, 4:pe19). Regarding the host effect of ERβ, it was reported that the B16 murine melanoma cell line grew significantly faster when injected into syngeneic ERβ knockout (KO) mice versus WT animals (Cho et al., Photochem Photobiol Sci 2010, 9:608-14). Three different syngenic murine tumor cell lines (mammary, colon, and melanoma) were injected separately into the newly established ERβ KI animals. Tumors of all three types grew significantly faster in KI animals than in WT controls (FIG. 21). These data support the notion that, in addition to its tumor-intrinsic effect demonstrated in published work, the same phosphotyrosine switch is important for host ERβ to inhibit tumor growth, thus furthering underscoring the physiological significance of the phosphotyrosine switch of ERβ.

B. Methods

Plasmids.

The expression vectors for ERα and ERβ (49) and GFP-fused c-Abl constructs (Tsai and Yuan, Cancer Res., 2003, 63(12):3418-24) were described previously. ERβ AF1 and AF2 deletion constructs were made by standard PCR. The Flag- and myc-tagged EYA2 were constructed using pcDNA3 (Invitrogen) and the pCDHEF1-MCS-T2A-Puro lentiviral expression vector (System Biosciences), respectively. The shRNA targeting sequence for EYA2, CATACCAACCTACTGCAGA (SEQ ID NO:3), was inserted into pSilencer2.1-U6neo (Ambion). Plasmids encoding GST fusion proteins were constructed in pGEX-KG (Amersham Biosciences). EYA2(D274A), EYA2(D502A) and ERβ Y36 mutations were generated by the QuickChange site-directed mutagenesis kit (Stratagene).

Cell Lines and Reagents.

Parental cell lines were purchased from American Tissue Culture Center (ATCC) and cultured per instructions from ATCC. Hs578T derivatives containing the doxycycline-inducible Flag-ER expression system were generously provided by Drs. John R. Hawse and Thomas C. Spelsberg. They were cultured in phenol red-free DMEM medium with 10% fetal bovine serum (FBS), supplemented with 5 mg/L blasticidin S (Invitrogen) and 500 μg/ml zeocin (Invitrogen). To establish stable cell pools with ectopic expression of EYA2 or ERβ, the corresponding lentiviruses were prepared in HEK293T cells and were used to infect various breast cancer cell lines. Stable cell pools or clones were established by selection in 2 μg/ml puromycin (Invitrogen). The HEK293 cells with inducible expression of WT and kinase-dead mutant c-Abl were previously described (Stuart et al., Oncogene, 2005, 24(55):8085-92). E2, PPT, and DPN were obtained from Tocris Inc. Imatinib mesylate and DPH were purchased from Selleck Chemicals (S1026) and Sigma-Aldrich (SML0202), respectively.

Antibodies.

The following antibodies are commercially available: anti-Flag M2 (A8592 and F3165, Sigma-Aldrich), anti-ERα (HC20, sc-543, Santa Cruz Biotechnology), anti-ERβ for immunoblotting (14C8, GeneTex; 9.88, Abcam), anti-ERβ for IP (EPR3777, Novus), anti-ERβ for IHC (68-4, Millipore), anti-EYA2 (HPA027024, Sigma-Aldrich), anti-pTyr (PY99, sc-7020, Santa Cruz Biotechnology), anti-p300 (sc-584, Santa Cruz Biotechnology Inc.), anti-GAPDH (G9295, Sigma-Aldrich), anti-FLAG-HRP (A8592, Sigma-Aldrich), anti-c-Abl (24-11, sc-23, Santa Cruz Biotechnology), and anti-Flag M2 agarose (A2220, Sigma-Aldrich). Two anti-pY36 antibodies were raised against an ERβ pY36-containing peptide SIYIPSS(pY)VDSHHE (SEQ ID NO:2), one in chicken by GenWay Biotech, Inc. and one in rabbit by Epitomics, Inc. Both were used interchangeably in Western blot analysis, and the rabbit antibody was used in IHC.

Yeast Two-Hybrid Screen.

The bait plasmid pGBKT7-ERβ(1-148) and a human mammary cDNA prey library (Clontech) were sequentially transformed into Saccharomyces cerevisiae strain AH109 according to the manufacturer's protocol. Transformants were grown on a synthetic medium lacking tryptophan, leucine, adenine, and histidine, but containing 1 mM 3-aminotriazole.

Co-IP and GST Pull-Down Assays.

Co-IP was performed as previously described (Aiyar et al., Genes & Dev., 2004, (18)2134-46). For GST pull-down assay, GST fusion proteins were expressed and purified according to the manufacturers' instructions (Amersham Pharmacia and Qiagen). ³⁵S-labeled, in vitro translated proteins were incubated with the GST fusion proteins bound to GST beads (Amersham Pharmacia), and the pull-down proteins were analyzed as previously described (Aiyar et al., Genes & Dev., 2004, (18)2134-46).

Transient Transfection.

All cells assessed for ligand stimulation were cultured in phenol red-free medium containing 5% charcoal stripped (CS) FBS for 3 days, re-seeded in 24-well Nunclon™ plates (Fisher), and transfected with various vectors as indicated in individual figures using Lipofectamine 2000 (Invitrogen) according to the manufacturer's instructions. Six hr after transfection, cells were treated with either vehicle or ligand at the indicated final concentration for 24 hr. For screening a human tyrosine kinase library (Addgene), individual kinase expression vectors was co-transfected with a myc-tagged ERβ expression vector into HEK293T cells. For the luciferase assay, renilla luciferase reporter vector phRL-SV40 (Promega) was used as an internal control. Luciferase values were normalized as described previously (Hu et al., J Biol Chem., 2000, (275)40910-915).

Real-Time RT-PCR.

RNA was extracted with Trizol reagent (Invitrogen). cDNA was synthesized with 1 μg of total RNA using the ImPromII Reverse Transcription System (Promega) and random primers. Quantitative PCR was conducted using the 7900HT Real-Time PCR System (Applied Biosystems). The level of GAPDH mRNA was measured as the internal control.

Chromatin Immunoprecipitation (ChIP).

For ChIP experiments in the ERβ-inducible Hs578T cells, 100 ng/ml doxcycline was added for 24 hr. Before harvest, cells were treated with either ethanol (vehicle) or 10 nM E2 for various time points. Cells were cross-linked with 1% formaldehyde for 10 min, treated with glycine at a final concentration of 0.125 M for 5 min at room temperature, and lysed in lysis buffer (5 mM HEPES pH 9.0, 85 mM KCl, 0.5% Triton X-100) for 15 min on ice. Nuclei were resuspended in nuclei lysis buffer (50 mM Tris-HCl pH 8.0, 10 mM EDTA, 1% SDS), and the cross-linked DNA was sonicated for 10 min (with a 30 s on/off cycle) using a Bioruptor sonicator (Diagenode). The supernatant was used for ChIP as previously described (Ding et al., Mol Cell., 2008, (31)347-59).

In Vitro Tyrosine Kinase Assay.

The assay was performed as described previously (Ding et al., J Clin Invst., 2009, 119(2):349-61). Briefly, Flag-ERβ was affinity-purified from transiently transfected HEK293T cells. WT and kinase-dead mutant Flag-c-Abl were inducibly expressed in HEK293 cells (Stuart et al., Oncogene, 2005, 24(55):8085-92) and purified in a similar fashion. ERβ and c-Abl proteins were then incubated in kinase buffer (50 mM HEPES, 10 mM MgCl₂, 1 mM DTT, 2.5 mM EGTA, 0.1 mM NA₃VO₃, 1 mM NaF), containing γ-³²P-ATP at 30° C. for 30 min. Kinase reactions were resolved by SDS-PAGE and exposed by autoradiography.

In Vitro Tyrosine Phosphatase Assay.

Commercially synthesized phospho-peptides or Flag-ERβ proteins immunoprecipitated from HEK293T cells were used as the substrates. The peptide sequences are as follows: pY142-H2AX (Krishnan et al., J Biol Chem., 2009, 284(24):16066-70): CPSGGKKATQASQE(pY)(SEQ ID NO:4); pY36-ERβ: SIYIPSS(pY)VDSHHE (SEQ ID NO:2). WT and mutant GST-EYA2 proteins were incubated with substrates in phosphatase buffer (50 mM Tris-HCl pH 7.0, 5 mM MgCl₂, 10% glycerol, 3 mg/ml BSA) at 37° C. for 30 min. Release of free phosphate was detected using Malachite Green detection assay per manufacturer's instruction (BIOMOL) (Cook et al., Nature, 2009, 458(7238):591-6).

Xenograft Assay.

45-day-old female athymic nude mice (Harlan) were injected orthotopically with MDAMB-231 cells into mammary gland fat pads. Tumor development was followed by caliper measurements along two orthogonal axes: length (L) and width (W). The volume (V) of tumors was estimated by the formula V=L×(W²)/2. All procedures involving animals and their care were approved by, and conducted in conformity with the guidelines of the Institutional Animal Care and Use Committee at the University of Texas Health Science Center at San Antonio.

Human Tissue Procurement and Analysis.

Rabbit anti-EYA2 (HPA027024, Sigma-Aldrich), anti-c-Abl (sc-887, Santa Cruz Biotechnology), anti-total ERβ (68-4, Millipore), and anti-pY36 antibodies were used as the primary antibodies for IHC. For analysis of correlation between pY36 and EYA2 or c-Abl, deidentified breast cancer samples were obtained with the informed consent of patients, following protocols approved by the Institutional Review Board from the Beijing Institute of Biotechnology and Affiliated Hospital of Chinese Academy of Military Medical Sciences. For the larger cohort study, the Breast Cancer Prognostic Tissue Microarrays (TMAs) were purchased from the National Cancer Institute Cancer Diagnosis Program. The TMAs contained 1169 non-metastatic breast tissue specimens divided into three TNM stages I-III. Immunohistochemistry of formalin-fixed paraffin-embedded samples was performed as described previously (Zhang et al., J Biol Chem., 2005, (280)43188-97). After staining, a total of 726 and 582 specimens from TMAs (age range, 25 to 96 year; mean±SD, 59.1±13.4; Median, 60) were available for analysis of pY36 and total ERβ, respectively. The other samples were either inadvertently detached from the case set during IHC or contained too few cells.

Each specimen was assigned a score according to the intensity of the nucleic and/or cytoplasmic staining (no staining=0; weak staining=1, moderate staining=2, strong staining=3) and the extent of stained cells (0%=0, 1-24%=1, 25-49%=2, 50-74%=3, 75-100%=4). The final immunoreactive score was determined by multiplying the intensity score with the extent of score of stained cells, ranging from 0 (the minimum score) to 12 (the maximum score). A score of 0 was defined as total ERβ-negative, pY36-negative, and EYA2-negative and score >1 as total ERβ positive, pY36-positive, and EYA2-positive, and score between 0-6 as c-Abl-negative and >6 as c-Abl-positive.

Statistical Analysis.

Statistical significance in the preclinical experiments was assessed by two-tailed Student's t test. The correlation between pY36 expression and clinic-pathologic characteristics was determined using Pearson's X² test. Disease-free survival was defined as the time from date of diagnosis to first recurrence (local or distant) or death from breast cancer without a recorded relapse. Overall survival was defined as the time from date of diagnosis to death where breast cancer was the primary or underlying cause of death. Patients who were alive at the last follow-up were censored at the last follow-up date, and patients who died from causes other than breast cancer were censored at the time of death. Estimation of disease-free survival and overall survival was performed using the Kaplan-Meier method, and differences between survival curves were determined with the log-rank test. All statistical tests were two-sided. Statistical calculations were performed using SPSS 13.0. In all assays, p<0.05 was considered statistically significant.

Oligonucleotides.

The c-Abl siRNA oligo sequences are: siAbl-1 GACAUCACCAUGAAGCACA (SEQ ID NO:5); siAbl-2 CUCCAUUGCUCCCUCGAAA (SEQ ID NO:6); siAbl-3 GCAACAAGCCCACUGUCUA (SEQ ID NO:7); siAbl-4 CCAGCUCUACUACCUACGU (SEQ ID NO:8).

Primers for RT-PCR and ChIP assays were designed by the Affymetrix Primer Express software program. Primer sequences are:

For mRNA analysis: MDA7 qF 5′-CTTTGTTCTCATCGTGTCACAAC-3′ (SEQ ID NO:9); MDA7 qR 5′-TCCAACTGTTTGAATGCTCTCC-3′(SEQ ID NO:10); MSMB qF 5′-CCAGGAGATTCAACCAGGAA-3′(SEQ ID NO:11); MSMB qR 5′-GAAACAAGGGTGCAACATGA-3′ (SEQ ID NO:12); NKG2E qF 5′-GCCAGCATTTTACCTTCCTCAT-3′ (SEQ ID NO:13); NKG2E qR 5′-AACATGATGAAACCCCGTCTAA-3′ (SEQ ID NO:14); HAVCR2 qF 5′-GAAGAAGAAGCAGTGACGGG-3′ (SEQ ID NO:15); HAVCR2 qR 5′-TGTCAGAATTGTGCTAGGCG-3′ (SEQ ID NO:16); PLA2G4D qF 5′-AGCCCCGGATCTGCTTTCT-3′ (SEQ ID NO:17); PLA2G4D qR 5′-GGTGAGGTCATACCAGGCATC-3′ (SEQ ID NO:18); pS2 qF 5′-CCCCGTGAAAGACAGAATTGT-3′ (SEQ ID NO:19); pS2 qR 5′-GGTGTCGTCGAAACAGCAG-3′ (SEQ ID NO:20); GREB1 qF 5′-CAAAGAATAACCTGTTGGCCCTGC-3′ (SEQ ID NO:21); GREB1 qR 5′-GACATGCCTGCCCTCTCATACTTA-3′ (SEQ ID NO:22); APOD qF 5′-GAATCAAATCGAAGGTGAAGCCA-3′ (SEQ ID NO:23); APOD qR 5′-ACGAGGGCATAGTTCTCATAGT-3′ (SEQ ID NO:24); WISP2 qF 5′-CCAACTCCACGTCTGCGAC-3′ (SEQ ID NO:25); WISP2 qR 5′-TCTCCCCTTCCCGATACAGG-3′ (SEQ ID NO:26); IGFBP4 qF 5′-GGTGACCACCCCAACAACAG-3′ (SEQ ID NO:27); and IGFBP4 qR 5′-GAATTTTGGCGAAGTGCTTCTG-3′ (SEQ ID NO:28).

(B) For ChIP analysis: MDA7 TSS F2 5′-CCCCATCGCTGTATTGT CCT-3′ (SEQ ID NO:29); MDA7 TSS R2 5′-GGAAAAAGAGGGAGGTGGAGA-3′ (SEQ ID NO:30); pS2 TSS F2 5′-GCTTAGGCCTAGACGGAATGGGC-3′ (SEQ ID NO:31); pS2 TSS R2 5′-CCAGGTCCTACTCATATCTGAGAG-3′ (SEQ ID NO:32); MSMB F1 5 ‘-GTCACTGGAAGGCACACAGA-3’ (SEQ ID NO:33); MSMB R1 5 ‘-CTTGTGCCAAGAAAGCCTGT-3’ (SEQ ID NO:34); GREB1 F2 5′-GCCAAATGGAAGAAGGACAG-3′ (SEQ ID NO:35); and GREB1 R2 5′-ACCACCTACCTCCAGTCACC-3′ (SEQ ID NO:36).

1. A method for evaluating the prognosis of a cancer patient comprising:
contacting a breast cancer sample from a patient with stage II or stage III breast cancer with an antibody that binds phosphorylated tyrosine 36 of estrogen receptor β:
quantifying phosphorylation of tyrosine 36 of ERβ; and
classifying the patient as having a good prognosis if phosphorylated tyrosine 36 levels are elevated or classifying the patient as having a poor prognosis if tyrosine levels are decreased relative to a non-cancer control.

2. The method of claim 1, further comprising measuring EYA2 or c-Abl activity.

3. The method of claim 1, wherein the cancer patient is a human.

4. The method of claim 1, wherein the test sample is aspirate or biopsy sample.

5. The method of claim 1, further comprising assessing the patient's clinical information.

6. The method of claim 5, wherein said clinical information comprises tumor size, tumor grade, lymph node status, and family history.

7. The method of claim 1, wherein said method for evaluating the prognosis of a breast cancer patient further comprises analyzing Her-2 expression levels in the breast cancer sample.

8. The method of claim 1, further comprising analyzing estrogen receptor or progesterone receptor status of the breast cancer patient.

9. A method for assessing the effectiveness of a cancer treatment comprising:
contacting a breast cancer sample from a patient with stage II or stage III breast cancer that has been administered an anti-cancer therapy with an antibody that binds phosphorylated tyrosine 36 of estrogen receptor β;
quantifying phosphorylation of tyrosine 36 of ERβ; and
classifying the treatment as effective if phosphorylated tyrosine 36 levels are elevated relative to levels prior to treatment.

10. An antibody that specifically binds phosphor-tyrosine 36 (pY36) of estrogen receptor β.

11. The antibody of claim 10, wherein the antibody binds a phosphopeptide having the amino acid sequence SIYIPSS(pY)VDSHHE and distinguishes between phosphorylated and unphosphorylated ERβ.

12. A kit for evaluating a breast cancer patient comprising the antibody of claim 9.


SEQUENCE LISTING (36 sequences)

SEQ ID NO: 1 - protein, 530 aa, homo sapiens
MDIKNSPSSLNSPSSYNCSQSILPLEHGSIYIPSSYVDSHHEYPAMTFYSPAVMNYSIPSNVTNLEGGPGRQTTSPNVLWPTPGHLSPLVVHRQLSHLYAEPQKSPWCEARSLEHTLPVNRETLKRKVSGNRCASPVTGPGSKRDAHFCAVCSDYASGYHYGVWSCEGCKAFFKRSIQGHNDYICPATNQCTIDKNRRKSCQACRLRKCYEVGMVKCGSRRERCGYRLVRRQRSADEQLHCAGKAKRSGGHAPRVRELLLDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHASNKGMEHLLNMKCKNVVPVYDLLLEMLNAHVLRGCKSSITGSECSPAEDSKSKEGSQNPQSQ

SEQ ID NO: 2 - protein, 14 aa, artificial
  peptide
SIYIPSSYVDSHHE

SEQ ID NO: 3 - DNA, 19 bp, Artificial Sequence
  synthetic oligonucleotide
cataccaacctactgcaga

SEQ ID NO: 4 - protein, 15 aa, Artificial Sequence
  synthetic peptide
CPSGGKKATQASQEY

SEQ ID NO: 5 - RNA, 19 nt, Artificial Sequence
  synthetic oligonucleotide
gacaucaccaugaagcaca

SEQ ID NO: 6 - RNA, 19 nt, Artificial Sequence
  synthetic oligonucleotide
cuccauugcucccucgaaa

SEQ ID NO: 7 - RNA, 19 nt, Artificial Sequence
  synthetic oligonucleotide
gcaacaagcccacugucua

SEQ ID NO: 8 - RNA, 19 nt, Artificial Sequence
  synthetic oligonucleotide
ccagcucuacuaccuacgu

SEQ ID NO: 9 - DNA, 23 bp, Artificial Sequence
  synthetic oligonucleotide
ctttgttctcatcgtgtcacaac

SEQ ID NO: 10 - DNA, 22 bp, Artificial Sequence
  synthetic oligonucleotide
tccaactgtttgaatgctctcc

SEQ ID NO: 11 - DNA, 20 bp, Artificial Sequence
  synthetic oligonucleotide
ccaggagattcaaccaggaa

SEQ ID NO: 12 - DNA, 20 bp, Artificial Sequence
  synthetic oligonucleotide
gaaacaagggtgcaacatga

SEQ ID NO: 13 - DNA, 22 bp, Artificial Sequence
  synthetic oligonucleotide
gccagcattttaccttcctcat

SEQ ID NO: 14 - DNA, 22 bp, Artificial Sequence
  synthetic oligonucleotide
aacatgatgaaaccccgtctaa

SEQ ID NO: 15 - DNA, 20 bp, Artificial Sequence
  synthetic oligonucleotide
gaagaagaagcagtgacggg

SEQ ID NO: 16 - DNA, 20 bp, Artificial Sequence
  synthetic oligonucleotide
tgtcagaattgtgctaggcg

SEQ ID NO: 17 - DNA, 19 bp, Artificial Sequence
  synthetic oligonucleotide
agccccggatctgctttct

SEQ ID NO: 18 - DNA, 21 bp, Artificial Sequence
  synthetic oligonucleotide
ggtgaggtcataccaggcatc

SEQ ID NO: 19 - DNA, 21 bp, Artificial Sequence
  synthetic oligonucleotide
ccccgtgaaagacagaattgt

SEQ ID NO: 20 - DNA, 19 bp, Artificial Sequence
  synthetic oligonucleotide
ggtgtcgtcgaaacagcag

SEQ ID NO: 21 - DNA, 24 bp, Artificial Sequence
  synthetic oligonucleotide
caaagaataacctgttggccctgc

SEQ ID NO: 22 - DNA, 24 bp, Artificial Sequence
  synthetic oligonucleotide
gacatgcctgccctctcatactta

SEQ ID NO: 23 - DNA, 23 bp, Artificial Sequence
  synthetic oligonucleotide
gaatcaaatcgaaggtgaagcca

SEQ ID NO: 24 - DNA, 22 bp, Artificial Sequence
  synthetic oligonucleotide
acgagggcatagttctcatagt

SEQ ID NO: 25 - DNA, 19 bp, Artificial Sequence
  synthetic oligonucleotide
ccaactccacgtctgcgac

SEQ ID NO: 26 - DNA, 20 bp, Artificial Sequence
  synthetic oligonucleotide
tctccccttcccgatacagg

SEQ ID NO: 27 - DNA, 20 bp, Artificial Sequence
  synthetic oligonucleotide
ggtgaccaccccaacaacag

SEQ ID NO: 28 - DNA, 22 bp, Artificial Sequence
  synthetic oligonucleotide
gaattttggcgaagtgcttctg

SEQ ID NO: 29 - DNA, 20 bp, Artificial Sequence
  synthetic oligonucleotide
ccccatcgctgtattgtcct

SEQ ID NO: 30 - DNA, 21 bp, Artificial Sequence
  synthetic oligonucleotide
ggaaaaagagggaggtggaga

SEQ ID NO: 31 - DNA, 23 bp, Artificial Sequence
  synthetic oligonucleotide
gcttaggcctagacggaatgggc

SEQ ID NO: 32 - DNA, 24 bp, Artificial Sequence
  synthetic oligonucleotide
ccaggtcctactcatatctgagag

SEQ ID NO: 33 - DNA, 20 bp, Artificial Sequence
  synthetic oligonucleotide
gtcactggaaggcacacaga

SEQ ID NO: 34 - DNA, 20 bp, Artificial Sequence
  synthetic oligonucleotide
cttgtgccaagaaagcctgt

SEQ ID NO: 35 - DNA, 20 bp, Artificial Sequence
  synthetic oligonucleotide
gccaaatggaagaaggacag

SEQ ID NO: 36 - DNA, 20 bp, Artificial Sequence
  synthetic oligonucleotide
accacctacctccagtcacc